SEQUENCE LISTING (8 sequences)

SEQ ID NO: 1 - DNA, 143 bp, artificial
  ICP4 gene fragment found in the chicken embryo origin vaccine
tactccccaccagaaagcttcacgtttcttccaacttctgcacaaaacttacgtaaccttgtcaatcggatcttgttctgcaggattctggaaatcgctagtccttccactagcattctatcatgtcctctgattcctcgacg

SEQ ID NO: 2 - DNA, 143 bp, artificial
  ICP4 gene fragment found in the tissue culture origin vaccine
tactccccaccagaaagcttcacgtttcttccaacttctgcacaaaacttacgtaaccttgtcactcgggtcttgttctgcaggattctggaaatcgctagtccttccactagcattctatcatgtcctctgattcctcgacg

SEQ ID NO: 3 - DNA, 143 bp, artificial; identical to SEQ ID NO: 1
  ICP4 gene fragment found in broiler and broiler/breeder isolates

SEQ ID NO: 4 - DNA, 143 bp, artificial
  ICP4 gene fragment found in the backyard flock isolate
tactccccaccagaaagcttcacgtttcttccaacttctgcacaaaacttacgtaaccttgtcaatcgggtcttgttctgcaggattctggaaatcgctagtccttccactagcattctatcatgtcctctgattcctcgacg

SEQ ID NO: 5 - DNA, 19 bp, artificial
  ICP4 non-coding region primer
acggtaatggtatgctggg

SEQ ID NO: 6 - DNA, 20 bp, artificial
  ICP4 coding region primer
ctcacagcggttgttttctc

SEQ ID NO: 7 - DNA, 20 bp, artificial
  ICP4 polymorphic site forward primer
tactactccccaccagaaag

SEQ ID NO: 8 - DNA, 21 bp, artificial
  ICP4 polymorphic site reverse primer
cgtcgaggaatcagaggacat

(19) United States
(12) Patent Application Publication
(10) Pub. No.: US 2010/0196881 A1
(43) Pub. Date: Aug. 5, 2010

(54) REVERSE RESTRICTION FRAGMENT LENGTH POLYMORPHISM ASSAY AND USES THEREOF

(75) Inventors: Scott A. Callison, Mocksville, NC (US); Maricarmen Garcia, Athens, GA (US); Sylva Riblet, Lexington, GA (US)

Correspondence Address: MUETING, RAASCH & GEBHARDT, P.A., P.O. BOX 581336, MINNEAPOLIS, MN, 55458-1336, US

(73) Assignee: University of Georgia Research Foundation, Inc., Athens, GA (US)

(21) Appl. No.: 12/353,545

(22) Filed: Jan. 14, 2009

Related U.S. Application Data
(63) Continuation-in-part of application No. PCT/US2007/016016, filed on Jul. 13, 2007.
(60) Provisional application No. 60/830,908, filed on Jul. 14, 2006.

(51) Int. Cl. C12Q 1/68

(52) U.S. Cl. 435/6

(57) ABSTRACT

The present invention presents a Reverse Restriction Fragment Length Polymorphism (RRFLP) method for the detection of the presence of an informative restriction enzyme site in a nucleotide sequence. The method includes digesting a sample with the informative restriction enzyme; performing polymerase chain reaction (PCR) on the digested sample with an oligonucleotide primer pair that flanks the informative restriction enzyme site; determining the Ct value of the sample; comparing the Ct value of the sample to the Ct value from a control sample; and calculating a ΔCt value, wherein a ΔCt value is the Ct value of the sample minus the Ct value of a control; and wherein a ΔCt value ≧+1 indicates that the informative restriction enzyme sites is present in the nucleotide sequence. The present invention includes the application of the RRFLP method for detection of the infectious laryngotracheitis virus (ILTV).

### CONTINUING APPLICATION DATA

This application is a continuation-in-part of International Application No. PCT/US 2007/016016, filed Jul. 14, 2007, which claims the benefit of U.S. Provisional Application Ser. No. 60/830,908, filed 14 Jul. 2006, all of which are incorporated herein by reference in their entireties.

### GOVERNMENT FUNDING

The present invention was made with government support under Grant Nos. 58-6612-2-219 and 10-21-RR188-174, awarded by the Agriculture Research Services, U.S. Department of Agriculture. The Government has certain rights in this invention.

### BACKGROUND

Traditional methods of genotyping ILTV isolates by multiple gene sequence analysis have many disadvantages. The genotype of a known viral isolate must be available for comparison and access to a sequencing facility is required. This method of genotyping viral isolates is time-consuming and costly. More recent methods of detecting the presence of viral infection using convention polymerase chain reaction (PCR) amplification have been developed. Although conventional PCR assays provide detection of ILTV with a high level of sensitivity and specificity, PCR assays do not give any information about the genotype of the ILTV in a particular sample. This information is important for epidemiological studies of the virus, which can be used to study the spread of the virus throughout poultry flocks. With conventional PCR, quantitative aspects are difficult and cumbersome to resolve. Furthermore, conventional PCR is prone to contamination and in some instances the interpretation of gel electrophoresis is inconclusive.

Another common molecular technique utilized for detection and differentiation of specific DNA sequences is the restriction fragment length polymorphism (RFLP) (Grodzicker et al., 1974 Cold Spring Harbor Symp. Quant; 39:439-446; Botstein et al, 1980, Am. J. Hum. Gen; 32:314-331). In this method, restriction enzymes are used to digest target DNA (genomic or PCR amplified), which is then separated by gel electrophoresis and visualized by staining. Comparing fragment patterns with known patterns or sequence data allows the determination of the presence or absence of specific sequences within the target DNA. This type of information has been used for the detection and differentiation of many different pathogens.

Conventional PCR amplification can be combined with a standard RFLP analysis in a two step PCR-RFLP assay. In this method, a specific genomic sequence harboring a specific and identifying polymorphism is amplified by PCR. Following gel purification and isolation, the amplicon is subjected to a standard RFLP technique. Many current methods of genotyping involve gene specific PCR followed by RFLP. See, for example, Chang et al., 1997, J Virol Meth; 66 (2):179-86; Clavijo et al., 1997, Avian Dis; 41 (1):241-6; Creelan et al., 2006, Avian Pathol; 35 (2):173-9; Han and Kim, 2003 Avian Dis; 47 (2):261-71; Han and Kim, 2001, Microbiol; 83 (4):321-31; Kirkpatrick et al., 2006, Avian Dis; 50 (1):28-34; Sellers et al., 2004, Avian Dis; 48 (2):430-6).

The assay methods of the present invention demonstrate an improvement over the conventional PCR, RFLP, and PCR-RFLP assays, demonstrating, for example, improved rapidity, sensitivity, reproducibility and the reduced risk of carry-over contamination, the simultaneous detection of viral infection and determination of the viral strain, a more objective interpretation of the RFLP analysis, utilization of small PCR products, and dramatically increased speed of the assay.

### SUMMARY OF THE INVENTION

The present invention includes a method of detecting the presence of a recognition site for a restriction enzyme in a nucleotide sequence, the method including: digesting all or a portion of a sample comprising the nucleotide sequence with the restriction enzyme; performing real-time polymerase chain reaction (PCR) on the sample digested with the restriction enzyme with an oligonucleotide primer pair that flanks the restriction enzyme recognition site; determining the Ct value of the sample digested with the restriction enzyme; comparing the Ct value of the sample digested with the restriction enzyme to the Ct value from a control sample not digested with the restriction enzyme; calculating a ΔCt value, wherein a ΔCt value is the Ct value of the sample digested with the restriction enzyme minus the Ct value of a control sample not digested with the restriction enzyme; wherein a ΔCt value of greater or equal to about +1 indicates that the nucleotide sequence is digested by the restriction enzyme at a recognition site located between the oligonucleotide primer pair.

In some embodiments of the method, separate portions of the sample are digested with different restriction enzymes and a separate ΔCt value is calculated for each separate portion.

In some embodiments, the method can be used in the detection and/or differentiation of strains of the avian pathogen infectious laryngotracheitis virus (ILTV). In some embodiments of the method, a portion of the sample is digested with the restriction enzyme Alw 1. In some embodiments of the method, a portion of the sample is digested with the restriction enzyme Ava 1. In some embodiments of the method a portion of the sample is digested with the restriction enzyme Ava I and a portion of the sample is digested with the restriction enzyme Alw 1. In some embodiments of the method, an oligonucleotide primer pair flank a region of about nucleotide 60 to about nucleotide 80 of the ILTV ICP4 gene promoter sequence may be used. In some embodiments of the method, the oligonucleotide primer pair is located within nucleotide positions 2039 to 2950 of the ILTV ICP4 gene (GENBANK Accession No. L32139). In some embodiments of the method, the oligonucleotide primer pair flanks nucleotide positions 2392 to 2534 of the ILTV ICP4 gene (GENBANK Accession No. L32139). In some embodiments of the method, the oligonucleotide primer pair is SEQ ID NO:7 and SEQ ID NO:8, or derivatives thereof. In some embodiments, the presence of a restriction enzyme site for Ava I and the absence of a restriction enzyme site for Alw 1 indicates the ILTV strain is related to the tissue culture origin (TCO) vaccine virus. In some embodiments, the presence of a restriction enzyme site for Alw I and the absence of a restriction enzyme site for Ava 1 indicates the ILTV strain is related to the chicken embryo origin (CEO) vaccine virus. In some embodiments, the method allows for the differentiation of an ILTV isolate into CEO-like, USDA/TCO-like, or wild-type.

The present invention also includes a method of detecting ILTV disease, the method including digesting a nucleotide sample with the restriction enzymes Alw 1 and/or Ava 1 and detecting the presence or absence of an Alw 1 and/or Ava 1 restriction. enzyme recognition site located about nucleotide 60 to about nucleotide 80 of the ILTV ICP4 gene promoter sequence.

The present invention includes oligonucleotide primers that flanks about nucleotide 60 to about nucleotide 80 of the ILTV ICP4 gene promoter sequence, including, but not limited to SEQ ID NO:7 and SEQ ID NO:8. The present invention also includes kits including one or more such primers.

The terms “comprises” and variations thereof do not have a limiting meaning where these terms appear in the description and claims.

Unless otherwise specified, “a,” “an,” “the,” and “at least one” are used interchangeably and mean one or more than one.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### BRIEF DESCRIPTION OF THE FIGURES

FIG. 1 is a schematic representation of the Reverse Restriction Fragment Length Polymorphism (RRFLP) assay of the present invention. A DNA sample is obtained, split into separate tubes and digested with specific restriction enzyme(s). The resultant DNA is used as a template for real-time PCR using primers that flank the specific restriction enzyme sites. Ct values of sample DNA exposed to restriction enzyme and are analyzed and compared to the Ct value of sample DNA not exposed to restriction enzyme (control). A ΔCt value is calculated and the results are analyzed.

FIG. 2 is a schematic representation of the standard PCR-based Restriction Fragment Length Polymorphism (PCR-RFLP) assay. A DNA sample is obtained and a specific target is amplified by PCR. The resultant amplicon is divided into separate tubes and digested with specific restriction enzyme(s). The resultant restriction fragments are loaded onto an agarose gel and the fragments are separated by gel electrophoresis, which sorts according to size. The fragments are visualized on the gel, for example, by staining with a DNA binding dye such as ethidium bromide or SYBR Green I. The banding pattern is analyzed for information.

FIG. 3 is a phylogenetic analysis of ICP4 5′ non-coding region encompassing nucleotide positions 2039 to 2950 (Accession number L32139). The phylogenetic tree was generated by the neighbor-joining method. The branch lengths represent the genetic distances between sequences, values are indicated in italics, in bold are bootstrap values indicated as a percentage at internal nodes (500 resamplings).

FIG. 4 shows polymorphic sites of the ICP4 gene fragment targeted by the RRFLP assay. The sequences presented correspond to nucleotide positions 2392 to 2534 of the ICP4 gene sequence (Accession # L32139). Included in the alignment are the sequences of the CEO (SEQ ID NO. 1) and TCO (SEQ ID NO. 2) vaccines, the sequence of the broiler (9/C/97/BR) and broiler breeder (23/H/01/BBR) isolates identified as BR/BBR (SEQ ID NO. 3), and the sequence of backyard flock isolate (24/H/91/BCK) identified as BCK (SEQ ID NO. 4). Shaded in light gray, in the CEO and BR/BRR sequences, is the Alw I enzyme recognition site. Shaded in dark gray, in the TCO sequence, is the Ava I enzyme recognition site. The backyard flock isolate lacked both restriction enzyme sites. Boxed nucleotides represent polymorphic sites recognized by restriction enzymes Alw I and Ava I.

FIGS. 5A to 5C are representative graphs obtained after RRFLP analysis. FIG. 5A presents Backyard flock isolate 24/H/91/BCK undigested C_{T} 25.6; digested Alw I C_{T} 25.97; digested Ava I C_{T} 25.64. FIG. 5B presents CEO vaccine undigested C_{T} 22.32; digested Alw I C_{T} 28.39; digested Ava I C_{T} 22.44. FIG. 5C presents TCO vaccine undigested C_{T} 26.01; digested Alw I C_{T} 26.05; digested Ava I C_{T} 30.58. The C_{T} value is calculated as the cycle number where the reaction fluorescence crosses the threshold line set at 10 units.

### DETAILED DESCRIPTION OF ILLUSTRATIVE EMBODIMENTS OF THE INVENTION

The present invention presents a method for the detection of the presence of informative restriction enzyme sites in a polynucleotide sequence. The method is a Reverse Restriction Fragment Length Polymorphism (RRFLP) method. This assay method has wide applicability, including, in particular, the field of molecular diagnostics. The method of the present invention can determine the presence or absence of an informative restriction enzyme site in a nucleotide sequence. Briefly, the method includes digesting all or a portion of a sample which includes the nucleotide sequence with the informative restriction enzyme; performing real-time polymerase chain reaction (PCR) on the sample digested with the restriction enzyme with an oligonucleotide primer pair that flanks the informative restriction enzyme site; determining the Ct value of the sample digested with the informative restriction enzyme; comparing the Ct value of the sample digested with the informative restriction enzyme to the Ct value from a control sample not digested with the informative restriction enzyme; and calculating a ΔCt value, wherein a ΔCt value is the Ct value of the sample digested with the informative restriction enzyme minus the Ct value of a control sample not digested with the informative restriction enzyme; wherein a ΔCt value of greater than or equal to about +1 indicates that the nucleotide sequence is digested by the informative restriction enzyme at a recognition site located between the oligonucleotide primer pair.

The RRFLP assay of the present invention may be applied in any situation where conventional Restriction Fragment Length (RFLP) is currently used. In RFLP, restriction enzymes are used to digest target DNA (genomic or PCR amplified), which is then separated by gel electrophoresis and visualized by staining. Comparing fragment patterns with known patterns or sequence data allows the determination of the presence or absence of specific sequences within the target DNA. This type of information has been used for the detection and differentiation of many different pathogens. See, for example, Grodzicker et al., 1974, Cold Spring Harbor Symp. Quant; 39:439-446; or Botstein et al., 1980, Am. J. Hum. Gen; 32:314-331. Conventional PCR amplification can be followed by a RFLP analysis in a two step PCR-RFLP assay. In PCR-RFLP, a specific genomic sequence harboring a specific and identifying polymorphism is amplified by PCR. Following gel purification and isolation, the amplicon is subjected to a standard RFLP technique.

The RRFLP method of the present invention has many advantages over conventional RFLP and PCR-RFLP assays. Gel electrophoresis is not required to obtain results. Instead, results are generated in real-time during a polymerase chain reaction (PCR). The RRFLP method of the present invention has wide application in molecular diagnostics, allowing for both the determination of the presence of a nucleic acid sequence in a sample and the genotyping of the nucleotide sample in the same assay. The RRFLP method of the present invention allows for a more objective interpretation that conventional RFLP analysis, utilizes small PCR products, and dramatically increases the speed of the RRFLP assay.

Real-time PCR (RT-PCR) may be used to monitor the formation of a double stranded DNA molecule in the RRFLP assay of the present invention. Recently, real-time PCR has become a fore runner of diagnostic detection methods. The reason for the rapid rise of real-time PCR is the extremely sensitive and specific nature of the method, along with its multiplex capabilities. First described by Higuchi et al., real-time PCR combines amplification with fluorometric detection of amplicons as the reaction occurs (Higuchi et al., 1993, Biotechnology; 11 (9):1026-1030). The ability to monitor the real-time progress of PCR has revolutionized PCR-based quantitation of DNA and RNA. The process of creating quantitative assays is streamlined because the construction and characterization of such standards are no longer required. Real-time PCR allows much more precise and reproducible quantitation of DNA and RNA than such methods as conventional PCR because it relies on CT values determined during the exponential phase of PCR rather then the endpoint. The concept of the threshold cycle (Ct) allows for accurate and reproducible quantification using fluorescence based RT-PCR. Fluorescent values are recorded during every cycle and represent the amount of product amplified to that point in the amplification reaction. The more templates present at the beginning of the reaction, the fewer number of cycles it takes to reach a point in which the fluorescent signal is first recorded as statistically significant above background, which is the definition of the (Ct) values. This will increase the throughput, because it is no longer necessary to analysis dilutions of each sample in order to obtain accurate results.

Any of the various means for implementing real-time PCR (RT-PCT) may be used in the present invention. For example, homogeneous detection of PCR products can be done using double-stranded DNA binding dyes, fluorogenic probes, and/or direct labeled primers. The detection of fluorescence during the thermal cycling process may, for example, be performed using Applied Biosystem's ABI Prism 7900 Sequence Detection Systems. For further discussion of real time PCR see the world wide web at ncifcrf.gov/rtp/gel/rtqpcr/WhatIs.asp; Higuchi et al., 1993, Biotechnology; 11 (9):1026-30; Mackay et al., 2002, Nucleic Acids Res; 30 (6):1292-305; and Mackay, 2004, Clin Microbiol Infect; 10 (3):190-212. In some embodiments, conventional PCR, rather that real-time PCR, may be used.

Real-time PCR detects products as they accumulate. A real-time system can utilize the intercalator ethidium bromide in each amplification reaction, an adapted thermal cycler to irradiate the samples with ultraviolet light, and detection of the resulting fluorescence with a computer-controlled cooled CCD camera. Amplification produces increasing amounts of double-stranded DNA, which binds ethidium bromide, resulting in an increase in fluorescence. By plotting the increase in fluorescence versus cycle number, the system produces amplification plots that provide a more complete picture of the PCR process than assaying product accumulation after a fixed number of cycles.

RT-PCR provides the ability to monitor the real-time progress of the PCR product via fluorescent detection. The point characterizes this in time during cycling when amplification of a PCR product is first detected rather than the amount of PCR product accumulated after a fixed number of cycles. These PCR-based fluorescent homogenous assays can be monitored by a variety of means, including, for example, labeled hybridization probe(s) (Taq Man, Molecular Beacons), labeled PCR primer (Amplifluor), and SYBR Green (Applied Biosystems).

As used herein, an informative restriction enzyme site reveals a pattern difference between the DNA fragment sizes in individual organisms after digestion with the restriction enzyme. To discover informative restriction enzyme sites, restriction enzymes (RE) are used to cut DNA at specific recognition sites. A restriction enzyme recognizes a specific recognition sequence of four to twelve nucleotides and cuts the DNA at a site within or a specific distance from the recognition sequence. For example, the restriction enzyme EcoRI recognizes the sequence GAATTC and cuts a DNA molecule between the G and the first A. Many different restriction enzymes are known and appropriate restriction enzymes can be selected for a desired result. Over 3,000 activities have been purified and characterized and more than 250 different sequence-specificities have been discovered. The recognition sequence of an informative restriction enzyme may be, for example, a four basepair sequence, a five basepair sequence, a six basepair sequence, an eight basepair sequence, or a twelve basepair sequence. A wide variety of such restriction enzymes are available. For a description of many restriction enzymes and their recognition sites and optimal buffer conditions see, for example, “Restriction Endonucleases Overview,” (available on the worldwide web at neb.com/nebecomm/tech_reference/restriction_enzymes/overview.asp) and the New England Biolabs 2007-2008 catalog. Informative restriction enzyme sites can be identified by digesting a sample DNA with one or more RE's and separating the resultant fragments according to molecular size using gel electrophoresis. Alternatively, informative restriction enzyme sites can be identified by analysis of genomic DNA sequences information.

Informative restriction enzyme sites can provide the basis for a single nucleotide polymorphism (SNP). A SNP is a DNA sequence variation occurring when a single nucleotide (A, T, C, or G) in the genome (or other shared sequence) differs between members of a species (or between paired chromosomes in an individual). For example, two sequenced DNA fragments from different individuals, AAGCCTA to AAGCTTA, contain a difference in a single nucleotide. In this case there are two alleles, C and T. If a restriction enzyme can be found such that it cuts only one possible allele of a section of DNA (that is, the alternate nucleotide of the SNP causes the restriction site to no longer exist within the section of DNA), this restriction enzyme is an informative restriction enzyme site that can be used in the RRFLP method of the present invention.

With the RRFLP method of the present invention, the informative restriction enzyme site is flanked by a pair of primers suitable for PCR analysis. Each of the oligonucleotide primers will hybridizes to the nucleotide sequence containing the informative restriction enzyme site and serve as primers for the PCR reaction. The ability to identify an oligonucleotide primer based upon a DNA sequence is described, for example, by Stein and Cohen (Cancer Res. 48:2659, 1988) and van der Krol et al. (BioTechniques 6:958, 1988). Oligonuclotide primers may be synthesized or may be obtained commercially.

It should be noted that any existing RFLP based detection/differentiation method that utilizes an informative (unique and specific) restriction enzyme site between two viable PCR primer sites could be easily converted to the RRFLP technique avoiding the use of more ambiguous and tedious gel based method for the differentiation of DNA molecules.

The RRFLP method of the present invention may be used in any system in which knowledge of one or more informative polymorphic restriction enzyme sites is available. Such systems includes, but are not limited to, applications in the fields of microbiology, virology, agriculture, plant genetics and breeding, medicine and veterinary medicine, forensic identification, paternity identification, pharmacogenetics, and diagnostic assays, for example diagnostic assays the detection of infectious diseases, genetic diseases, and cancer. Examples of the application of the RRFLP method of the present invention, include, but are not limited to, those discussed in more detail below. In some embodiments of the present invention, separate portions of the sample may be digested with different informative restriction enzymes and a separate ΔCt value calculated for each separate portion.

The RRFLP method of the present invention may be used for the detection and characterization of infectious laryngotrachetis virus (ILTV) based on the amplification of a 222-base-pair PCR fragment using primers located in a conserved region of the infected cell protein 4 gene that encompasses a single nucleotide polymorphism restriction endonuclease MspI. Creelan et al. described this polymorphism in more detail (Creelan et al., 2006, Avian Pathol; 35 (2):173-9).

The RRFLP method of the present invention may be used for the identification and differentiation of varicella-zoster virus (VZV) wild-type strains from the attenuated varicella Oka vaccine strain based on the PCR amplification of a VZV open reading frame (ORF) 62 region. A single specific amplicon of 268 by with a SmaI enables accurate strain differentiation; there are three SmaI sites present in amplicons of vaccine strain VZV, compared with two enzyme cleavage sites for all other VZV strains tested). Thus, the Oka vaccine strain can be accurately differentiated from wild-type VZV strains circulating in countries representing all six populated continents. Moreover, this informative restriction enzyme site reliably distinguishes wild-type Japanese strains from vaccine strains (Loparev et al., 2000, J Clin Microbiol; 38 (9):3156-60).

The RRFLP method of the present invention may be used for differentiation between field isolates and live vaccine strains ts-11 and 6/85 of Mycoplasma gallisepticum (“MG”) in Israel. PCR primers targeted to the gene mgc2, encoding a cytadherence-related surface protein are uniquely present in MG. The mgc2-PCR diagnostic primers are specific for MG in tests of all avian mycoplasmas or bacteria present in the chicken trachea and are sensitive enough to readily detect MG in tracheal swabs from field outbreaks. Differentiation of vaccine strain ts-11 is based on restriction enzyme sites in the 300-base-pair (bp) mgc2-PCR amplicon present in ts-11 and missing in MG isolates from field outbreaks in Israel. Restriction sites for the enzymes HaeII and SfaN1 are present in the amplified region in strain ts-11 and not in twenty-eight field isolates of MG, comprising a representative cross section of all the MG isolates from the period 1997-2003; mgc2-PCR amplification and restriction of the amplicon with HaeII, gives a 270-bp fragment for ts-11 or no restriction for other MG strains tested. This test can also be used to identify the 6/85 vaccine strain, which yields a 237-bp product, readily differentiated from the approximately 300-bp PCR product of all other strains tested. Lysnyansky et al. describes these polymorphisms in more detail (Lysnyansky et al., 2005, Avian Dis; 49 (2):238-45 and 49 (3):451).

The RRFLP method of the present invention may be used for the differentiation of Mycoplasma pulmonis and Mycoplasma arthritidis. Digestion with the restriction enzyme Sma I is coupled with the use of a genus-specific sequence of mycoplasma for the PCR reaction. Four isolates of M. pulmonis contain an informative Sma I restriction site, while there was no digestion with Sma I in M. arthritidis. Kim et al. describes this polymorphism in more detail (Kim et al., 2005, Exp Anim; 54:359-62).

The RRFLP method of the present invention may be used for the detection of a polymorphism at codon 129 of the prion protein gene that has been shown to confer genetic susceptibility to prion diseases and to influence the epidemic course of variant Creutzfeldt-Jakob disease. This polymorphism is described in more detail by de Paula et al. (de Paula et al., 2005, Eur J Epidemiol; 20:593-5).

The RRFLP method of the present invention may utilize any of the many RFLPs that serve as genetic markers of disease, identifying whether or not an individual possesses a particular genetic defect. There is growing list of inherited disorders where DNA probes are available to test whether or not someone is carrying a defective gene. Some examples include, but are not limited to, Duchenne muscular dystrophy where progressive muscle weakness is caused by a genetic deficiency in dystrophin protein (for further info see the world wide web at mdausa.org); cystic fibrosis where respiratory functioning is impaired and is related to genetic deficiency in a membrane ion channel protein (see the world wide web at cff.org); blood disorders such as hemophilia and sickle cell anemia caused by testable genetic defects; and Tay-Sachs disease.

The RRFLP method of the present invention may utilize any of the various RFLPs that serve as a genetic marker for cancer. Such markers for breast cancer, including the BRCA1 gene, prostate cancer, and colon cancer are described, for example, by Watkins, 1988 Biotechniques 6 (4):310-319, 322. The RRFLP method of the present invention may be used for any of a variety of applications in DNA fingerprinting, such as to identify genetic diversity within breeding populations in plants and animals, to differentiate between plant species cultivars, as well as to identify plants containing a gene of interest.

The RRFLP method of the present invention may be used in human identity applications, such as forensic analysis in crimes where DNA samples of suspects are amplified for typing experiments against samples taken from the scene of the crime.

The RRFLP method of the present invention has application in the rapidly developing fields of pharmacogenetics and personalized medicine in which genetically determined propensities of individual patients to respond favorably or adversely to a given pharmacologic agent can be determined prior to administration of that drug (Cartwright, 2001 Expert Rev Mol Diagn. 1 (4):371-6).

The RRFLP method of the present invention may be used to identify and differentiate strains of various poultry pathogens. Embodiments of the RRFLP method of the present invention may be used in a wide range of rapid diagnostic assays, including, for example, assays for mycoplasmosis, infectious bronchitis, and infectious laryngotracheitis. The RRFLP method of the present invention may be used to identify and differentiate poultry pathogens such as Campylobacter, infectious bursal disease virus, Newcastle disease virus, infectious bronchitis virus, Mycoplasma gallisepticum, fowl adenovirus, Salmonella, and avian parvoviruses. For more detail on these pathogens, see, for example, Ayling et al., 1996, Res. Vet. Sci; 60 (2):168-172; Jackwood and Sommer, 1997, Avian Dis; 41 (3):627-637; Kou et al., 1999, J. Vet. Med. Sci ; 61 (11):1191-1195; Kwon et al., 1993, Avian Dis; 37 (1):194-202; Lysnyansky et al., 2005, Avian Dis; 49 (2):238-245; Meulemans et al., 2004, Avian Pathol; 33 (2):164-170; Park et al., 2001, J. Vet. Sci; 2 (3):213-219; and Sirivan et al., 1998, Avian Dis; 42 (1):133-139.

The RRFLP method of the present invention may be used to identify and differentiate the avian pathogen infectious laryngotracheitis virus (ILTV), a member of the family Herpesviridae, subfamily alphaherpesviridae. Infectious laryngotracheitis (ILT) is an upper-respiratory disease of poultry of worldwide distribution (Guy and Bagust, 2003 Diseases of Poultry, Iowa State University Press: Ames, Iowa; pp. 121-134) characterized by acute respiratory signs, which include gasping, coughing, sneezing, depression, nasal discharge, and conjunctivitis. For severe forms of the disease, signs include labored breathing and expectoration of bloody mucous, while severe hemorrhages and mucous plugs are observable upon gross examination of the trachea. Although some severe signs of the disease are characteristic, in less severe episodes of the disease many signs are similar to other acute respiratory diseases of poultry (Linares et al., 1994, Avian Dis; 38:188-92; Sellers et al., 2004, Avian Dis; 48 (2):430-6) and the need for a specific differential diagnosis is essential for the rapid detection of the virus.

The disease is common in areas of intense poultry production and during severe outbreaks it causes great economic losses due to high bird morbidity and moderate mortality. The disease is mainly controlled by vaccination with live attenuated vaccines. Traditionally in the U.S. two types of live-attenuated vaccines have been widely utilized. One type of vaccine is attenuated by multiple passages in embryonated eggs (chicken embryo origin, “CEO”) (Samberg et al. 1971, Avian Dis; 15:413-417). In the second type, vaccine is generated by multiple passages in tissue culture of chicken cells (tissue culture origin, “TCO”) (Gelenczei & Marty, 1965 Avian Dis. 14:44-56).

Infectious laryngotracheitis continues to emerge in the field on a regular basis in poultry producing states. Evidence is mounting that most field outbreaks are caused by viruses indistinguishable from chicken-embryo-origin vaccine strains, and for that reason, broiler outbreaks are often referred to in the field as “vaccinal laryngotracheitis” (VLT). There is a need for improved, rapid diagnostic methods for identifying ILTV strains, including VLT strains (Dufour-Zavala, 2008, Avian Dis; 52:1-7).

In detection, the virus can be isolated from field material in specific pathogen free (SPF) chicken embryos (CE) inoculated via the chorioallantoic (CAM) route or by isolation in primary chicken embryo kidney (CEK), chicken embryo liver (CELi), or in chicken kidney (CK) cells. Although sensitive, virus isolation may take three to four weeks and several cell culture passages are required before the cytopathic effect (CPE) caused by ILTV replication appears in the cell culture systems. Therefore, rapid assays for detection of the virus are usually performed in combination with virus isolation to accelerate the diagnosis of the disease. Histopathology examination remains the standard method for the rapid diagnosis of ILT. Characteristic lesions of ILT include syncytial cell formation of the tracheal epithelial cells with intranuclear inclusion bodies, necrosis, and hemorrhage. Inclusion bodies are present only during the early stages of infection (one to seven days post infection) and disappear as infection progresses as a result of the necrosis and desquamation of epithelial cells. And in cases of mild forms of the disease the differential diagnosis based on histopathological differentiation can be difficult due to infrequent number of intranuclear inclusions found with or without syncytial cell formation. Therefore other rapid assays have been utilized for the detection of ILTV such as the use of fluorescently-labeled polyclonal antibodies (FA) as immunoprobes to detect viral antigens intracheal and conjunctival smears. Monoclonal antibodies have been used to detect viral antigens in frozen tracheal sections by an indirect immunoperoxidase method, and in an antigen capture ELISA.

Earlier experimental evidence demonstrated that live attenuated vaccine strains could easily revert to virulence after bird-to-bird passage (Guy et al., 1991, Avian Dis; 35 (2):348-355), or after reactivation from latency (Hughes et al., 1991, Arch. Virology; 121:213-218). Once vaccine strains have been introduced in the field the identification of ILTV strains is difficult because of the antigenic and genomic homogeneity of the vaccines and field viruses (Guy and Bagust, 2003, Diseases of Poultry, Iowa State University Press: Ames, Iowa; pp. 121-134). Initial attempts to differentiate among ILTV strains in the U.S. were achieved by restriction fragment length polymorphism (RFLP) analysis of the viral genome (Leib et al., 1986, Avian Dis; 30:835-837; Guy et al., 1989, Avian Dis; 33:316-323; Andreasen et al., 1990, Avian Dis; 34:646-656; Keller et al., 1992, Avian Dis; 36:575-581; Keeler et al., 1993, Avian Dis; 37:418-426). However, routine use of RFLP analysis of the viral genome for epidemiological purposes was limited due to the difficulties in obtaining high yields of pure viral DNA.

With the advent of the polymerase chain reaction, restriction fragment length polymorphism of PCR products (PCR-RFLP) has greatly facilitated the differentiation of ILTV strains. PCR-RFLP and sequencing analysis of single and multiple viral genes and genome regions has permitted the differentiation of ILTV isolates from vaccine strains in different parts of the world (Chang et al, 1997, J Virol. Meth; 66:179-186; Clavijo and Nagy, 1997, Avian Dis; 41 (1):241-246; Graham et al., 2000, Avian Pathol; 29:57-62; Han and Kim, 2001, Vet. Microbiol; 4:321-331; Han and Kim, 2003, Avian Dis; 47:261-271; Kirkpatrick et al., 2006, Avian Dis; 50:28-34; Creelan et al., 2006, Avian Pathol; 35 (2):173-179). The use of multiple gene sequence analysis and multiple PCR-RFLP has been essential to identify informative single nucleotide polymorphism (SNP) appropriate for the discrimination of isolates from a particular region (Kirkpatrick et al., 2006, Avian Dis; 50:28-34, Ojkic et al., 2006, Avian Pathol; 35 (4):286-292, Oldoni and Garcia 2007, Avian Pathol; 36 (2):167-176). PCR-RFLP analysis of informative SNPs has been utilized to differentiate field isolates form vaccine strains (Creelan et al., 2006, Avian Pathol; 35 (2):173-179).

The RRFLP assay of the present invention may be applied as a method for the detection of ILTV infected birds. Indeed, PCR has already proven to be an effective and rapid test to detect ILTV infected birds in severe (Williams et al., 1992, J Gen Virol; 73 (9):2415-20) and mild outbreaks (Sellers et al., 2004, Avian Dis; 48 (2):430-6) of the disease. And, PCR has been successfully used to detect ILTV DNA from trachea scrapings of experimentally and naturally infected chickens, from extra-tracheal sites such as the conjuctiva and from the trigeminal ganglia and from formalin-fixed, paraffin-embedded tissues as well.

The RRFLP assay of the present invention may be used as a means of detecting specific informative polymorphic sites in the avian infectious laryngotracheitis virus (ILTV) genome. During the RRFLP procedure, DNA is digested with restriction enzymes targeting an informative polymorphic site and then used as template in a real-time polymerase chain reaction (PCR) with primers flanking this region. The analysis of the ΔC_{T} values obtained from digested and undigested template DNA provides the genotype of the DNA. In the examples described herein, the RRFLP assay was applied as a method to differentiate between the two types of infectious laryngotracheitis virus attenuated live vaccines. Sequence analysis of ILTV vaccines revealed an informative polymorphic site in the 5′ non-coding region of the infected cell protein (ICP4) present in the tissue culture origin (TCO) and chicken embryo origin (CEO) attenuated vaccines recognized by restriction enzymes AvaI and AlwI, respectively. These two informative polymorphic sites were used in a RRFLP assay to rapidly and reproducibly genotype ILTV attenuated live vaccines.

The RRFLP assay of the present invention may be applied as a method to differentiate between the two different types of infectious laryngotracheitis virus attenuated live vaccines. Sequence analysis of ILTV vaccines revealed an informative polymorphic site in the 5′ non-coding region of the infected cell protein (ICP4) recognized by restriction enzymes AvaI and AlwI present in the tissue culture origin (TCO) and chicken embryo origin (CEO) attenuated vaccines, respectively. These two informative polymorphic sites were used in a RRFLP assay to rapidly and reproducibly genotype ILTV attenuated live vaccines, outbreak related isolates, and clinical isolates.

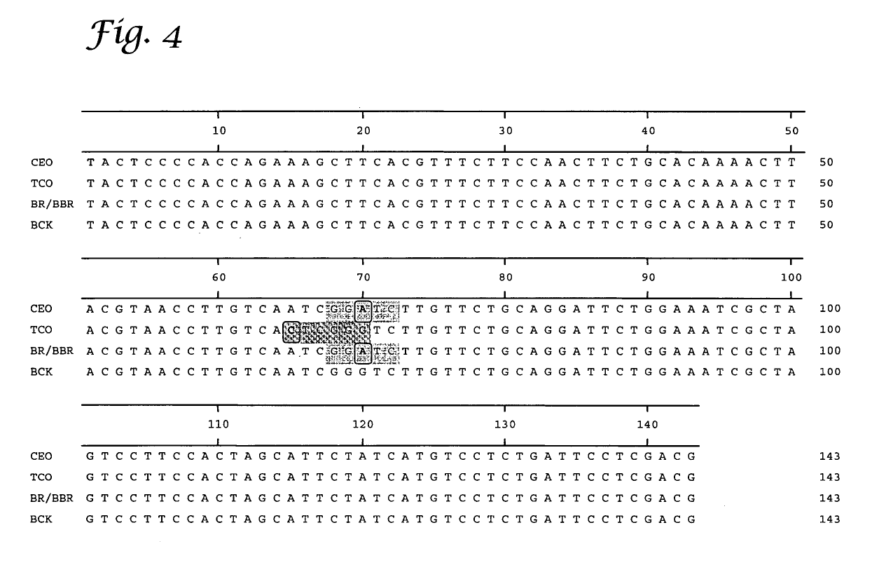

The methods of the present invention may be used in the detection and/or differentiation of ILTV strains. In some embodiments of the present invention, for the detection and/or differentiation of ILTV strains, and/or a portion of the sample may be digested with the restriction enzyme Alw 1 and a portion of the sample may be digested with the restriction enzyme Ava I and the oligonucleotide primer pair flanks about nucleotide 60 to about nucleotide 80 of the ICP4 gene promoter sequence (as shown in FIG. 4).

Application of the RRFLP method of the present invention include, but are not limited to, those described in more detail in the Examples included herewith. The RRFLP assay of the present invention can be used as a novel diagnostic assay for the differentiation of infectious laryngotracheitis virus isolates. The method of the present invention has commercial applicability, for example, in the poultry diagnostic laboratory as a rapid means of differentiating an ILTV isolate into one of three categories, wild type, CEO vaccine or TCO vaccine.

The present invention provides kits for detecting or differentiating ILTV. Such kits may include one or more of the following: one or more primers, one or more restriction enzymes, buffer, one or more polymerases, buffer, and dNTPs. Such kits may include oligonucleotide primers that flank an informative restriction enzyme site in the ICP4 gene (Accession No. L32139).

The complete nucleotide sequence of the infectious laryngotracheitis virus (ILTV) gene encoding a homologue to the ICP4 protein of herpes simplex virus (HSV) has been determined. The ILTV ORF encoding ICP4 is 4386 nucleotides long, calculated from the first of four ATG codons, and has an overall G+C content of 59%. The ILTV ICP4 contains two domains of high homology which have been reported in other studies to be conserved in the ICP4 homologues of alpha herpes viruses, and to be functionally important. Several regulatory features were identified including a serine-rich domain in region one. A more extensive serine-rich domain was located in region five which is also found in varicella-zoster virus (VZV) and bovine herpesvirus 1. For more detail, see Johnson et al., 1995, Virus Res; 35 (2):193-204. Accession No. L32139 is the complete nucleotide sequence, bases 1 to 8364, of the major immediate early protein (ICP4) gene of infectious laryngotracheitis virus (gallid herpesvirus 1) (version L32139.1, Feb. 27, 1996), and is incorporated by reference herein. The sequence is available on the world wide web at ncbi.nlm.nih.gov/entrez/viewer.fcgi?db=nuccore&id=493597.

With the present invention, informative restriction enzyme sites are located between about nucleotide 60 to about nucleotide 80 of the ICP4 gene promoter sequence and in SEQ ID NO:1, SEQ ID NO:2, SEQ ID NO:3, or SEQ ID NO:4, as shown in FIG. 4. The oligonucleotide primer pair may be located within nucleotide positions 2039 to 2950 of the ICP4 gene (Accession No. L32139). The oligonucleotide primer pair may flank nucleotide positions 2392 to 2534 of the ICP4 gene (GENBANK Accession No. L32139). The oligonucleotide primers may flank about nucleotide 60 to about nucleotide 80 of the ICP4 gene promoter sequence SEQ ID NO:1, SEQ ID NO:2, SEQ ID NO:3, or SEQ ID NO:4, as shown in FIG. 4. One of the oligonucleotide primers may include SEQ ID NO: 7, consist of SEQ ID NO:7, or may be a sequence derived from or hybridizable to SEQ ID NO:7. One of the oligonucleotide primers may include SEQ ID NO:8, consist of SEQ ID NO:7, or may be a sequence derived from or hybridizable to SEQ ID NO:8. A kit of the present invention may also include the restriction enzyme Alw 1, and/or the restriction enzyme Ava I, and/or restriction buffers for the Alw 1 and/or Ava 1 restriction enzymes. The kit may include printed instructions, one or more positive controls, one or more negative controls, and/or reagents used in the performance of PCR, such as, for example, 10× buffers and/or polymerase enzymes.

The present invention includes isolated oligonucleotide primers for use in the methods of the present invention. Oligonucleotide primers of the present invention may flank the informative Alw 1 or Ava 1 restriction enzymes sites found within nucleotide positions 2039 to 2950 of the ICP4 gene (Accession No. L32139), or this site in SEQ ID NO:1, SEQ ID NO:2, 2039 to 2950 of SEQ ID NO:3, or SEQ ID NO:4. Oligonucleotide primers of the present invention include, but are not limited to, any of the oligonucleotide primers described herein. Oligonucleotide primers of the present invention may be found within nucleotide positions 2039 to 2950 of the ICP4 gene (Accession No. L32139), or be complementary to a sequence within nucleotide positions 2039 to 2950 of the ICP4 gene (Accession No. L32139). An oligonucleotide primer of the present invention may flank nucleotide positions 2392 to 2534 of the ICP4 gene (GENBANK Accession No. L32139) or this site in SEQ ID NO:1, SEQ ID NO:2, SEQ ID NO:3, or SEQ ID NO:4. An oligonucleotide primer of the present invention may flank about nucleotide 60 to about nucleotide 80 of the ICP4 gene promoter sequence SEQ ID NO:1, SEQ ID NO:2, SEQ ID NO:3, or SEQ ID NO:4, as shown in FIG. 4. An oligonucleotide primer of the present invention may be a forward primer that includes SEQ ID NO: 7 or may be a sequence derived from or hybridizable to SEQ ID NO:7. An oligonucleotide primer of the present invention may be a reverse primer that includes SEQ ID NO:8 or may be a sequence derived from or hybridizable to SEQ ID NO:8. The present invention also includes an isolated oligonucleotide primer having the sequence ACGGTAATGGTATGCTGGG (SEQ ID NO:5), CTCACAGCGGTTGTTTTCTC (SEQ ID NO:6), TACTACTCCCC ACCAGAAAG (SEQ ID NO:7), or CGTCGAGGAATC AGAGGACAT (SEQ ID NO:8). The present invention also includes an isolated oligonucleotide primer selected from the group consisting SEQ ID NO:5, SEQ ID NO:6, SEQ ID NO:7, and SEQ ID NO:8.

An isolated primer of the present invention may be a nucleic acid sequence that hybridizes under stringent conditions to one or more of nucleotides 2039 to 2950 of the ICP4 gene (Accession No. L32139); SEQ ID NO:1; SEQ ID NO:2; SEQ ID NO:3; SEQ ID NO:4; the direct complement of nucleotides 2039 to 2950 of the ICP4 gene (Accession No. L32139); the direct complement of SEQ ID NO:1; the direct complement of SEQ ID NO:2; the direct complement of SEQ ID NO:3; or the direct complement of SEQ ID NO:4.

The term hybridization refers to the process in which one single-stranded polynucleotide non-covalently binds in a base-specific manner to a second complementary strand of nucleic acid to form a double-stranded polynucleotide. The resulting double-stranded polynucleotide is a “hybrid.” Methods for conducting polynucleotide hybridization assays have been well developed in the art. Hybridization assay procedures and conditions will vary depending on the application and are selected in accordance with the general binding methods known including, for example, those referred to in: Maniatis et al. Molecular Cloning: A Laboratory Manual (3rd Ed. Cold Spring Harbor, N.Y., 2002). Hybridizations may be performed under stringent conditions, for example, at a salt concentration of no more than 1 M and a temperature of at least 25° C. As used herein, stringent hybridization conditions may be 50% formamide, 5×SSC, 50 M sodium phosphate (pH 6.8), 0.1% sodium pyrophosphate, 5× Denhardt's solution, sonicated salmon sperm DNA (50 μg/ml, 0.1% SDS, and 10% dextran sulfate at 42° C., with washes at 42° C. in 0.2×SSC and 50% formamide at 55° C., followed by a wash comprising of 0.1×SSC containing EDTA at 55° C. Further, conditions of 5×SSPE (750 mM NaCl, 50 mM NaPhosphate, 5 mM EDTA, pH 7.4) and a temperature of 25-30° C. may be suitable for allele-specific probe hybridizations. See, for example, Sambrook et al., (2001).

With the RRLFP method of the present invention, one primer is located at each end of the region to be amplified. Such primers may be between about 10 to about 30 nucleotides in length, about 15 to about 25 nucleotides in length, or about 18 to about 22 nucleotides. The smallest sequence that can be amplified is approximately 50 nucleotides in length (e.g., a forward and reverse primer, both of 20 nucleotides in length, whose location in the sequences is separated by at least 10 nucleotides). Much longer sequences can be amplified. In some embodiments, the length of sequence amplified is between about 75 and about 250 nucleotides in length.

Primers of the present invention may be designed to hybridize near a polymorphism so that the polymorphism is between the site of primer binding and a restriction site. In one embodiment polymorphisms and priming sites are selected so that the distance between the restriction site and the primer binding site is within about 200, about 500, about 1000 or about 2000 base pairs.

One primer is called the “forward primer” and is located at the left end of the region to be amplified. The forward primer is identical in sequence to a region in the top strand of the DNA (when a double-stranded DNA is pictured using the convention where the top strand is shown with polarity in the 5′ to 3′ direction). The sequence of the forward primer is such that it hybridizes to the strand of the DNA which is complementary to the top strand of DNA. The other primer is called the “reverse primer” and is located at the right end of the region to be amplified. The sequence of the reverse primer is such that it is complementary in sequence to a region in the top strand of the DNA. The reverse primer hybridizes to the top strand of the DNA.

PCR primers may be chosen subject to a number of other conditions. PCR primers should be long enough (preferably about 10 to about 30 nucleotides in length) to minimize hybridization to greater than one region in the template. Primers with long runs of a single base should be avoided, if possible. Primers may preferably have a percent G+C content of between 40 and 60%. If possible, the percent G+C content of the 3′ end of the primer should be higher than the percent G+C content of the 5′ end of the primer. Primers should not contain sequences that can hybridize to another sequence within the primer (i.e., palindromes). Two primers used in the same PCR reaction should not be able to hybridize to one another. Although PCR primers are preferably chosen subject to the recommendations above, it is not necessary that the primers conform to these conditions. Other primers may work.

PCR primers that can be used to amplify DNA within a given sequence may be chosen using one of a number of computer programs that are available. Such programs choose primers that are optimum for amplification of a given sequence (for example, such programs choose primers subject to the conditions stated above, plus other conditions that may maximize the functionality of PCR primers). One such computer program is the Genetics Computer Group (GCG recently became Accelrys) analysis package which has a routine for selection of PCR primers. There are also several web sites that can be used to select optimal PCR primers to amplify an input sequence. Examples of such web sites are alces.med.umn.edu/rawprimer.html and genome.wi.mit.edu/cgi-in/primer/primer3_www.cgi.

An oligonucleotide primer of the present invention may be about 5 to about 50 nucleotides in length, about 14 nucleotides to about 30 nucleotides in length, about 17 to about 23 nucleotides in length, about 18 to about 22 nucleotides in length, about 19 to about 21 nucleotides in length. It may be about 5 bases in length, about 6 bases in length, about 7 bases in length, about 8 bases in length, about 9 bases in length, about 10 bases in length, about 11 bases in length, about 12 bases in length, about 13 bases in length, about 14 bases in length, about 15 bases in length, about 16 bases in length, about 17 bases in length, about 18 bases in length, about 19 bases in length, about 20 bases in length, about 21 bases in length, about 22 bases in length, about 23 bases in length, about 24 bases in length, about 25 bases in length, about 30 bases in length, about 35 bases in length, about 40 bases in length, about 45 bases in length, or about 50 bases in length.

As used herein an “isolated” nucleic acid molecule or primer is identified and separated from at least one contaminant nucleic acid molecule with which it is ordinarily associated in the natural source of the nucleic acid. An isolated nucleic acid molecule is other than in the form or setting in which it is found in nature.

Samples that can be used in the methods of the present invention can be obtained from any source. Samples include, but are not limited to, environmental or food samples and medical or veterinary samples. Samples may be liquid, solid, or semi-solid. Samples may be swabs of solid surfaces. Samples may include environmental materials, such as the water samples, airborne particles such as pollen and dust, and filters from air samples. Samples may be of meat, poultry, processed foods, milk, cheese, or other dairy products. Samples may be foodstuffs, beverages, cosmetic products, pharmaceutical products, healthcare products, or surfaces such as floors and tables. Medical or veterinary samples include, but are not limited to, blood, blood products, tissue, ascites, culture media, body fluids, skin, pus, urogenital specimens, feces, sputum, cerebrospinal fluid, fecal samples, and different types of swabs. A sample may be obtained from a clinical isolates, for example, and isolate obtained from skin or soft tissue infections. A sample may be obtained from a swab of a body site, for example, from the nose, including, but not limited to, the anterior nares, the throat, the perineum, the axilla, or the skin. A sample may be obtained from an individual. An individual, may be, for example, an avian species, such as, for example, poultry, chickens, ducks, and turkeys, mammals, such as, for example, a human, dogs, cats, cow, pig, horse, mouse, hamster, and plants, yeast, bacteria, and fungi. Samples may be used directly in the methods of the present invention, without preparation or dilution. Samples may be diluted or suspended in solution, which may include, but is not limited to a buffered solution or a bacterial culture medium. A sample that is a solid or semi-solid may be suspending in a liquid by mincing, mixing or macerating the solid in the liquid.

Various embodiments of the method of the present invention may include any of a variety of steps, including, but not limited to, for example, obtaining or providing a nucleic acid sample; isolating genomic DNA from a sample; determining the presence or absence of an informative restriction site in a polynucleotide sample; contacting a polynucleotide sample with a primer pair; providing a primer set for amplifying a DNA fragment; adding a primer pair to the sample, wherein said primer pair binds to a target DNA sequence in the sample; amplifying said target DNA sequence; detecting an amplification product generated using a oligonucleotide primer pair; and/or identifying in a nucleic acid sample, a nucleotide occurrence of a single nucleotide polymorphism (SNP).

With the present invention, DNA for RRFLP analysis may be prepared by any of a variety of methods, including, but not limited to any of those described herein. For example, extraction by a standard procedure such as that described in Ausubel, F. M., R. Brent, R. E. Kingston, B. D. Moore, J. G. Seidman, J. A. Smith, and K. Struhl. 1987. Current protocols in molecular biology. Greene Publishing Associates and Wiley Interscience, New York, N.Y. may be used.

The present invention also includes methods of detecting or genotyping infectious laryngotracheitis virus (ILTV) in a sample by identifying the presence or absence of the informative Alw 1 or Ava 1 restriction enzymes sites found within nucleotide positions 2039 to 2950 of the ICP4 gene (Accession No. L32139) using any of a variety of methods in addition to RRFLP, such as, for example, Southern analysis of genomic DNA, direct mutation analysis by restriction enzyme digestion, Northern analysis of RNA, denaturing high pressure liquid chromatography (DHPLC), gene isolation and sequencing, hybridization of a specific oligonucleotide probe with amplified gene products, conventional RFLP assays, and/or PCR-RFLP assays.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals.

The present invention is illustrated by the following examples. It is to be understood that the particular examples, materials, amounts, and procedures are to be interpreted broadly in accordance with the scope and spirit of the invention as set forth herein.

### Example 1

### Genotyping Infectious Laryngotracheitis Virus (ILTV) by Multiple Gene Sequencing and Reverse Restriction Fragment Length Polymorphism (RRFLP)

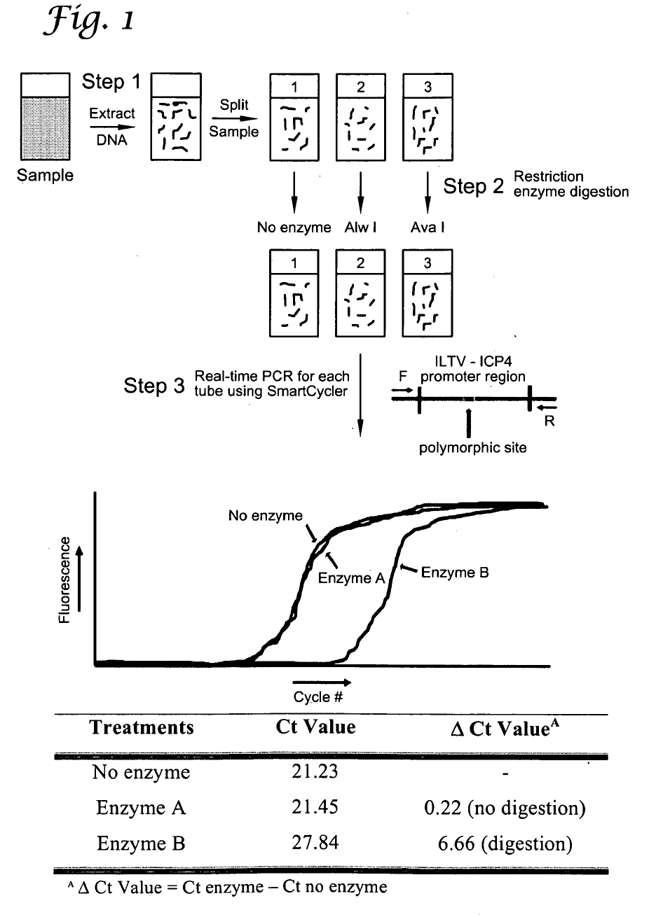

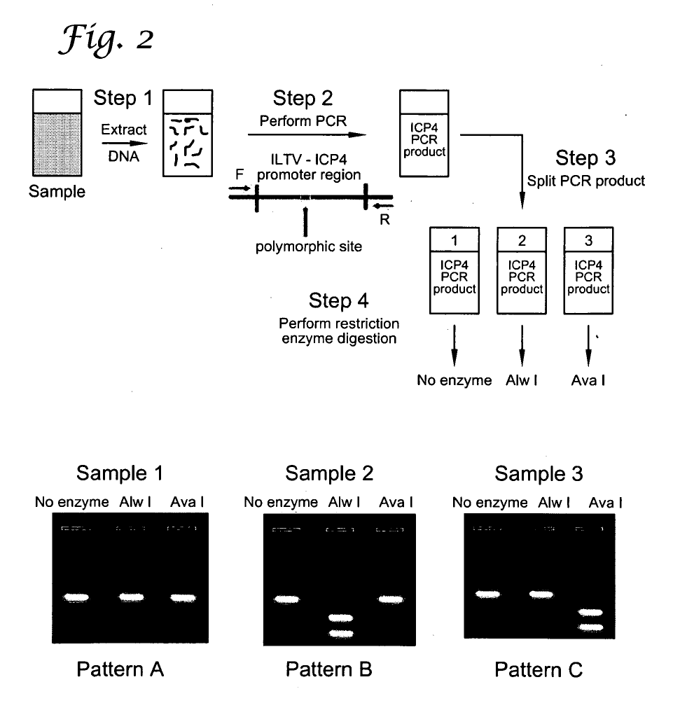

In this example, the development of a novel Reverse Restriction Fragment Length Polymorphism method for the identification of infectious laryngotracheitis virus genotype is described. In order to understand the Reverse Restriction Fragment Length Polymorphism (RRFLP) assay of the present invention (FIG. 1), it is constructive to compare the conventional Restriction Fragment Length Polymorphism (RFLP) assay for determining viral genotypes (FIG. 2).

Infectious laryngotracheitis virus (ILTV) is an acute respiratory disease of chickens that affects poultry worldwide. Waves of the disease are observed in US one or twice a year particularly in areas of dense broiler production. In an effort to better understand the origin of these outbreaks typing of outbreak-related isolates has been conducted by multiple viral gene sequencing. The construction of a database of viral sequences from vaccine strains, backyard flock isolates, broiler and breeder isolates led to the differentiation of viral strains and the identification of genome sites that can be utilized as makers to trace isolates in the field. Using this multiple gene sequence typing approach, 27 ILTV isolates originated between the years 1991 to 2005 from different regions in the US, from broilers, layers, broiler-breeders and backyard flocks were characterized (Table 1). Sequencing analysis of multiple genes has allowed the differentiation of US ILTV isolates into three main groups CEO-like (23 isolates), TCO-like (1 isolate), and field or backyard-flock isolates (3 isolates).

Although conventional polymerase chain reaction (PCR) assays provide detection of ILTV with a high level of sensitivity and specificity, such assays do not give any information about the genotype of the ILTV in a particular sample. This information is important for epidemiological studies of the virus, which can be used to study the spread of the virus throughout poultry flocks. Current methods of genotyping involve the two steps of gene specific PCR followed by RFLP (Chang et al., 1997, J Virol Methods; 66 (2):179-86; Clavijo et al., 1997, Avian Dis; 41 (1):241-6; Creelan et al., 2006, Avian Pathol; 35 (2):173-9; Han and Kim, 2003, Avian Dis; 47 (2):261-71; Han and Kim, 2001, Microbiol; 83 (4):321-31; Kirkpatrick et al., 2006, Avian Dis; 50 (1):28-34; Sellers et al., 2004, Avian Dis; 48 (2):430-6) or sequence analysis (Chang et al., 2000, Avian Dis; 44 (1):125-31; and Han and Kim, 2001, Microbiol; 83 (4):321-31).

TABLE 1
Genotype of United States ILTV strains
by multiple gene sequencing analysis.

| Isolates | Origin |
|---|---|
| 648-88 | BYF^{A} |
| S2816-02 | BYF |
| 8954-97 | BRF^{B} |
| 9030-97 | BRF |
| 7227-03 | BRF |
| 7253-03 | BRF |
| 7271-03 | BRF |
| 7399-03 | BRF |
| 32396-03 | BRF |
| 32650-03 | BRF |
| 18138-04 | BBF |
| JEL-04 | BRF |
| K0400820-04 | BRF |
| L6172-04 | BRF |
| L6811B-04 | BRF |
| L6811C-04 | BRF |
| L15505-04 | BRF |
| S1687A-04 | BRF |
| S1687B-04 | BRF |
| CL1-04 | BRF |
| CL2-04 | BRF |
| PA1-04 | LYF^{C} |
| 1219-00 | BBF^{D} |
| 6902-02 | BBF |
| Chicken embryo origin (CEO) | Vaccine |
| Tissue culture origin (TCO) | Vaccine |
| USDA | Challenge strain |

^{A}BYF—backyard flock;
^{B}BRF—broiler flock;
^{C}BBF—broiler breeder flock;
^{D}layer flock

However, with conventional PCR, quantitative aspects are difficult and cumbersome to resolve. Furthermore, conventional PCR is prone to contamination and in some instances the interpretation of gel electrophoresis is inconclusive. As the second generation of nucleic amplification methods, real-time PCR has received wider acceptance in the diagnosis of viral diseases due to its improved rapidity, sensitivity, reproducibility and the reduced risk of carry-over contamination (Mackay et al., 2002, Nucleic Acids Res; 30 (6):1292-305). In addition, the capability of real time PCR to quantify viral DNA has added clinical value to the molecular diagnostic of viral diseases.

Rather than PCR-RFLP, a novel technique named Reverse Restriction Fragment Length Polymorphism (RRFLP) assay was utilized for analyses. The RRFLP assay is a novel technique where the viral DNA is initially digested with restriction enzymes followed by real-time polymerase chain reaction (PCR) amplification of the target genome region. The analysis of the cycle threshold (ΔC_{T}) differences obtained from digested and undigested template DNA provides the genotype of the sample. The Ct value is the PCR cycle number where a particular reaction crosses a predefined threshold level of fluorescence. To calculate the ΔCt value, the following formula is used:

ΔCt value=(Ct value of reaction exposed to restriction enzyme)−(Ct value of reaction not exposed to restriction enzyme).

For any sample, a ΔCt value≧+1 after treatment with a specific restriction enzyme means that the sample DNA is susceptible to cleavage by the specific restriction enzyme at a site between the flanking primer set. Therefore (FIG. 1), the sample was digested by Enzyme B between the flanking primers, which meant that there was a site between the flanking primer set susceptible to restriction enzyme B and this information differentiated the sample into a particular genotype.

### Example 2

### Reverse Restriction Fragment Length Polymorphism (RRFLP) Assay: A Novel Technique and its Application for the Rapid Genotyping of Infectious Laryngotracheitis Virus (ILTV) Live Attenuated Vaccines

Molecular based assays are becoming more common as the standard for diagnostic detection and differentiation of many pathogens. Mainly due to the increased sensitivity, and specificity of molecular assays, real-time PCR has become the forerunner of diagnostic detection methods due to its extreme sensitivity and specificity and multiplex capabilities. First described by Higuchi et al., real-time PCR combines amplification with fluorometric detection of amplicons as the reaction occurs (Higuchi et al., 1993, Bio/Technology; 11:1026-1030).

Another common molecular technique utilized for the rapid differentiation of specific DNA sequences is the restriction fragment length polymorphism (RFLP). In this method, restriction enzymes are used to digest target DNA (genomic or PCR amplified), which is then separated by gel electrophoresis and visualized by staining. This technique has been used for the detection and differentiation of many pathogens; particularly has been widely utilized in poultry diseases to identify strains from different poultry pathogens (Jackwood M. W. and D. Jackwood, in: Swayne, D. E., Glisson J. R., Jackwood, M. W., Pearson, J. E., Reed W. M. (Eds.), Isolation and identification of avian pathogens 5^{th} edition, 2008. American Association of Avian Pathologist, University of Pennsylvania, New Bolton Center, PA). One of these pathogens is infectious laryngotracheitis virus (ILTV), a member of the family Herpesviridae, subfamily alphaherpesviridae. Infectious laryngotracheitis (ILT) is an upper-respiratory disease of poultry of worldwide distribution (Guy and Garcia, “Infectious laryngotracheitis virus,” in: Saif, Y. M., Glisson, J. R., Fadly, A. M., McDougald, L. R., Nolan, L. K., Swayne, D. E. (Eds.), Diseases of Poultry 12^{th} edition, 2007, Blackwell Publishing Inc., Ames, Iowa) characterized by acute respiratory signs, which include gasping, coughing, sneezing, depression, nasal discharge, and conjunctivitis. The disease is common in areas of intense poultry production and during severe outbreaks it causes great economic losses due to high bird morbidity and moderate mortality. The disease is mainly controlled by vaccination with live attenuated vaccines. Traditionally in the U.S. two types of live-attenuated vaccines have been widely utilized; these vaccines has been attenuated by multiple passages in embryonated eggs (chicken embryo origin, “CEO”) (Samberg and Aronovici, 1969, Refuah Veterinarith; 26:54-59); and the vaccine generated by multiple passages in tissue culture of chicken cells (tissue culture origin, “TCO”) (Gelenczei and Marty, 1964, Avian Dis; 8:105-122).

Once vaccine strains have been introduced in the field the identification of ILTV strains is difficult because of the antigenic and genomic homogeneity of the vaccines and field viruses (Guy and Garcia, “Infectious laryngotracheitis virus,” in: Saif, Y. M., Glisson, J. R., Fadly, A. M., McDougald, L. R., Nolan, L. K., Swayne, D. E. (Eds.), Diseases of Poultry 12^{th} edition, 2007. Blackwell Publishing Inc., Ames, Iowa). Initial attempts to differentiate among ILTV strains in the U.S. were achieved by RFLP analysis of the viral genome (Leib et al., 1986, Avian Dis; 30:835-837; Guy et al., 1989, Avian Dis; 33:316-323; Andreasen et al., 1990, Avian Dis; 34:646-656; Keller et al., 1992, Avian Dis; 36:575-581; and Keeler et al., 1993, Avian Dis; 37:418-426). However, routine use of RFLP analysis of the viral genome was limited due to the difficulties of obtaining high yields of pure viral DNA. With the advent of the polymerase chain reaction, PCR-RFLP and sequencing analysis of single and multiple genome regions has permitted the differentiation of ILTV isolates from vaccine strains in different parts of the world (Chang et al., 1997, J. Virol. Meth; 66:179-186; Clavijo and Nagy, 1997, Avian Dis; 41:241-246; Graham et al., 2000, Avian Pathol; 29:57-62; Han and Kim, 2001, Vet. Microbiol; 4:321-331; Han and Kim, 2003, Avian Dis; 47:261-271; Kirkpartick et al., 2006, Avian Dis; 50:28-34; and Creelan et al., 2006, Avian Pathol; 5 (2):173-179). The use of multiple gene sequence analysis and multiple PCR-RFLP has been essential to identify informative single nucleotide polymorphism (SNP) capable of differentiate field isolates form vaccine strains (Kirkpartick et al., 2006, Avian Dis; 50:28-34; Ojkic et al., 2006, Avian Pathol; 35 (4):286-292; Oldoni et al., 2007, Avian Pathol; 36:167-176; and Creelan et al., 2006, Avian Pathol; 5 (2):173-179).

In this example, two SNPs were identified, and utilized to differentiate between ILTV live attenuated vaccines (CEO and TCO). Rather than PCR-RFLP, reverse restriction fragment length polymorphism (RRFLP) assay was utilized in the analysis. The RRFLP is a technique where the viral DNA is initially digested with restriction enzymes followed by real-time polymerase chain reaction (PCR) amplification of the target genome region. The analysis of the cycle threshold (ΔC_{T}) differences obtained from digested and undigested template DNA provides the genotype of the sample. This example demonstrates the first use of the RRFLP assay and its application to rapidly differentiate between ILTV attenuated live vaccines utilized in the U.S.

### Materials and Methods

ILTV strains and isolates. Six commercially available chicken embryo origin (CEO) ILTV vaccines were obtained from Intervet America (Millsboro, Del., USA), Lohman Animal Health (Winslow, Mass., USA), Schering-Plough Animal Health (Omaha, Nebr., USA), and Fort Dodge (Fort Dodge, Iowa, USA). The tissue culture origin (TCO) ILTV vaccine was obtained from Schering-Plough Animal Health (Omaha, Nebr., USA), and the USDA reference strain of ILTV was obtained from the American Type Culture Collection (ATCC) (Manassas, Va., USA). All vaccines were re-suspended in 10 ml of sterile phosphate buffered saline (PBS) and aliquots were stored at −80° C. as stocks for further propagation. Each vaccine strain was propagated and passed three consecutive times in chicken kidney (CK) cells prepared as previously described (Tripathy and Garcia, “Laryngotracheitis” in, Swayne, D. E., Glisson, J. R., Jackwood, M. W., Pearson, J. E., Reed, W. M. (Eds.), Isolation and identification of avian pathogens 5^{th} edition, American Association of Avian Pathologist, University of Pennsylvania, New Bolton Center, PA). Viral isolates selected for this study were obtained from broiler, broiler-breeder, and backyard flock outbreaks (Table 2). Viral isolates were propagated in the chorioallantoic membrane (CAM) of chicken embryos, and the second passage in CAM was utilized for viral DNA extraction.

DNA Extraction. DNA extraction from viral isolates, trachea, and eye conjunctiva samples was performed using the Qiamp Mini kit (Qiagen, Valencia, Calif.) with modifications from the manufacturer's recommendations. Briefly, 100 μl of the swab suspension was incubated with 10 μl of proteinase K and 400 μl of lysis buffer (AL) at 56° C. for 10 minutes. After incubation, 100 μl of 100% ethanol was added to the lysate. The samples were then washed and centrifuged following the manufacturer's recommendations. Nucleic acid was eluted with 100 μl of elution buffer provided in the kit.

Amplification and Sequence Analysis of the ICP4 Gene Fragment. A 1,246 base pair fragment of the ICP4 gene, encompassing part of the gene non-coding region and portion of the 5′ coding region was amplified and sequenced for six CEO vaccines, the TCO vaccine and ten ILTV field isolates. The amplification and sequencing reactions were performed using primers listed in Table 3. The PCR reaction was assembly as follows: 28.5 μl of water, 5 μl of 10×PCR buffer, 4 μl of 25 mM MgCl₂, 5 μl of 1 mM dNTPs, 1 μl of 5 μM of ICP4 non-coding primer, 1 μl of 5 μM ICP4 coding primer, 0.5 μl of Taq polymerase (5 U/μl), and 5 μl of template. The reaction was cycled in a conventional thermocycler using a program of 1 cycle of 94° C., 2 minutes; 35 cycles of 94° C., 1 minute; 53° C., 1 minute; 72° C., 1.5 minutes; and 1 cycle of 72° C., 12 minutes. After amplification, the PCR products were separated by gel electrophoresis on a 1.0% agarose gel and visualized by UV trans-illumination. Amplification reactions producing the expected 1,246 base pair fragment were purified with a QIAquick PCR purification kit (Qiagen, Valencia, Calif.). Purified amplification products were sequenced using the amplification primers (Table 3). Raw sequence data was edited using SeqMan and aligned using MegAlign programs from DNASTAR software version 6.0 (DNASTAR, Inc. Madison, Wis.). Percentage sequence identity of the ICP4 sequences was determined by clustal method and phylogenetic analysis was performed using the neighbor joining group method and 500 bootstrap resampling using the PAUP Version 4 program (Sinauer Associates, Inc. Publishers).

Real-Time (ReTi) ILTV PCR Assay. Before RRFLP analysis the viral genome copy number log₁₀ per sample was determined by real time PCR ILTV assay as previously described (Callison et al., 2007, J. Virol. Meth; 139:31-38). Briefly, the primers and probe are located in the viral glycoprotein C and were synthesized by EDT (Coralville, Iowa), and BioSearch Technologies (Novato, Calif.). The final reaction volume was 25 μl including; 12.5 μliters of 2× master mix (Quantitect Probe PCR kit, Qiagen, Valencia, Calif.), primers were utilized to a final concentration of 0.5 μmolar, probe to a final concentration of 0.1 μmolar, 1 μl of HK-UNG (Epicentre, Madison, Wis.), 2 μl of water, and 5 μl of DNA template. The tubes were closed and cycled in a Smart Cycler thermocycler (Cepheid, Sunnyvale, Calif.) using a thermocycle program of 50° C., 2 minutes; 95° C., 15 minutes; and 40 cycles of 94° C., 15 seconds; 60° C., 60 seconds with optics ON. To determine the genome copy number log₁₀, per sample a standard curve was generated and the equation previously reported (Callison et al., 2007, J. Virol. Meth; 139:31-38) was utilized for quantification of viral genomes copy number found per sample.

Reverse Restriction Fragment Length Polymorphism (RRFLP) Analysis. Vaccine strains, outbreak-related isolates, and clinical samples (trachea and eye conjunctiva) with C_{T} values≦30.00, as determined by the ReTi assay, were analyzed by RRFLP. Briefly, each DNA sample was divided into three separate aliquots of 5 μl. Viral genome digestions were performed with Alw I and Ava I. Each digestion reaction was set up as follows: Tube 1 (no enzyme control)—5.0 μliters of DNA, 1 μliters of 10× reaction buffer, and 4 μliters of water; Tube 2 (Alw I enzyme)—5.0 μliters of DNA, 1 μliter of 10× reaction buffer, 1 μliter of restriction enzyme, and 3 μliters of water; Tube 3 (Ava I enzyme)—5.0 μliters of DNA, 1 μliter of 10× reaction buffer, 1 μliter of restriction enzyme, and 3 μliters of water. Digestions were performed at 37° C. for 2 hours followed by 80° C. for 20 minutes. After digestion, 90 μliters of distilled water were added to each tube. The diluted DNA from the digested (Alw I and Ava I) and the undigested (no enzyme control) was used as template in real-time PCR. Primers flanking the two SNPs (Table 3) were designed to amplify a 146 base pair product using 12.5 μliters of 2× master mix (Quantitect SYBR Green I PCR kit, Qiagen, Valencia, Calif.), primers to a final concentration of 0.5 μmolar, 5.5 μliters of water, and 5 μliters of DNA template. The tubes were closed and cycled in a Smart Cycler thermocycler (Cepheid, Sunnyvale, Calif.) using a thermocycle program of 95° C., 15 minutes; and 40 cycles of 94° C., 15 seconds; 60° C., 30 seconds; 72° C., 30 seconds with optics ON. For each reaction, the threshold cycle number (C_{T} value) was determined to be the PCR cycle number at which the fluorescence of the reaction exceeded 10 units of fluorescence in the FAM channel. The background minimum and maximum cycle values were set to 5 and 15, respectively. The RRFLP results were recorded as the difference of the threshold cycle number (ΔC_{T}) obtained from the C_{T} of the Alw I and the Ava I digestion reactions minus the C_{T} value from the undigested (uncut) reaction. Samples with ΔC_{T} values for Alw I digestion ≧1, and ΔC_{T} value for Ava I digestion ≦1 were genotyped as CEO vaccine virus; and samples with a ΔC_{T} value for Alw I digestion reaction of ≦1, and a ΔC_{T} value for Ava I digestion of ≧1 were genotyped as TCO like virus; and samples with ΔC_{T} values ≦1 for either restriction enzyme were genotyped as wild-type strains.

Vaccination Experiments. In order to evaluate the reproducibility of the RRFLP analysis two separate experiments with CEO and TCO vaccinated chickens, and chickens in contact exposure to vaccinated birds were performed. Ninety-six white leghorn specific pathogen free (SPF) chickens were obtained from Merial (Gainesville, Ga.) for each experiment. Chickens were housed in stainless steel cages in an isolation room with filtered-air and positive-pressure at the Poultry Diagnostic and Research Center (PDRC, Athens, Ga.), and fed a standard diet and water ad libitum. At four weeks of age, birds were divided in four groups of twenty-four chickens per cage, 12 of which were vaccinated, and 12 were contact-exposed to the vaccinated birds. Wing bands were used in to identify contact-exposed birds. Chickens were vaccinated by eye-drop with the TCO and CEO live attenuated vaccine during two separate experiments using the recommended dose (33 μl per chicken). Larynx/trachea swabs were collected from two vaccinated and two contact-exposed chickens at 2 and 4, 5 to 10, 14, and 18 days post-vaccination in 1 ml of sterile phosphate buffered saline solution (PBSS) containing antibiotic-antimycotic 100× (Gibco, Grand Island, N.Y.) and 2% newborn calf serum (Gibco Grand Island, N.Y.). Tracheal samples were storage at −80 C until further PCR processing.

### Results

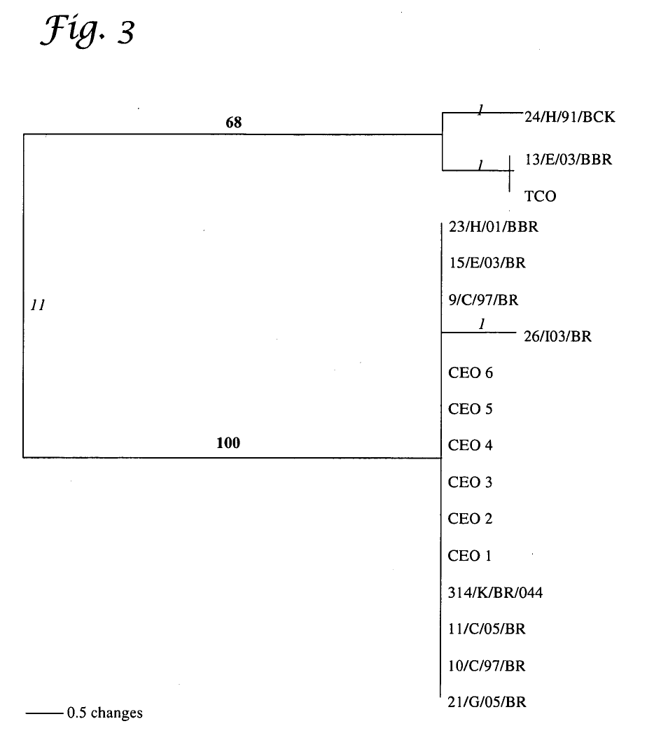

Sequencing analysis of ICP4 gene fragment. Sequence analysis corresponding to nucleotide positions 2039 to 2950 of the ICP4 gene (Accession number L32139) was performed for CEO vaccines, TCO vaccine, and 10 field isolates (Table 2). Sequences were separated in two main lineages (FIG. 3), a lineage that includes the six commercial CEO vaccines and eight outbreak related isolates with 99.9 to 100% sequence similarity. A second lineage containing the TCO vaccine, one back-yard flock isolate (24/H/91/BCK), and one broiler-breeder isolate (13/E/03/BBR). The breeder isolate (13/E/03/BBR) sequence was 100% similar to the TCO vaccine and share 99.8% similarity with the back-yard flock isolate (24/H/91/BCK) sequence. An informative polymorphic site, with two SNPs, was identified in the 5′ non-coding region of the ICP4 gene (FIG. 4). Within this region, the CEO vaccine strains and isolates, 9/C/97/BR, 10/C/97/BR, 23/H/01/BBR, 15/E/03/BR, 26/1/03/BR, 11/C/05/BR, 21/G/05/BR, 314/K/BR/04 (identified in FIG. 4 as BR/BBR) contained a unique Alw I restriction site. The TCO vaccine strain contained a unique Ava I restriction site, and the backyard flock strain 24/H/91/BCK lacked either restriction enzyme site.

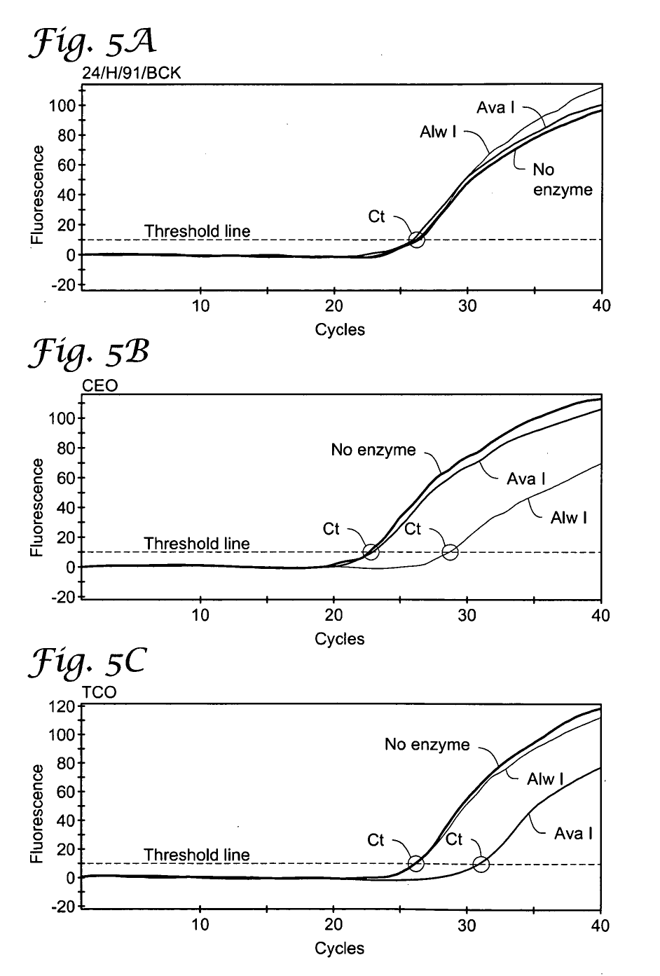

Reverse Restriction Fragment Length Polymorphism (RRFLP). The RRFLP technique was developed around two SNPs that constitute the informative polymorphic site (FIG. 4). The RRFLP assay interpretation is shown in FIG. 5A-5C. The CEO vaccine, TCO vaccine, and the backyard flock isolate 24/H/91/BCK were selected for initial analysis. Backyard flock isolate (24/H/91/BCK) lacked both restriction enzyme sites as determined by sequencing analysis, and confirmed by the ΔC_{T} values of 0.32 for Alw I and −0.01 for Ava I (FIG. 5A). The CEO strain was susceptible to Alw I digestion (ΔC_{T} of 6.07) and resistant to Ava I digestion (ΔC_{T} value of 0.12) indicating that the CEO type viruses has the Alw I site and lacks the Ava I site (FIG. 5B). The TCO strain was susceptible to the Ava I digestion (ΔCt value of 4.57) while resistant to Alw I digestion (ΔC_{T} value of 0.04) indicating that the viral nucleic acid has the Ava I site and lacks the Alw I (FIG. 5C).

Analysis of vaccine strains and field isolates by RRFLP. A total seven vaccines, 10 field isolates, and two CK cells passages of CEO and TCO vaccines were analyzed by RRFLP of the polymorphic sites and sequencing analysis of the complete ICP4 5′ non-coding region. Based on ΔC_{T} values, the RRFLP assay typed isolates 9/C/97/BR, 10/C/97/BR, 23/H/01/BBR, 15/E/03/BR, 26/I/03/BR, 11/C/05/BR, 21/G/05/BR, 314/K/BR/04 as CEO, isolate 13/E/03/BBR as TCO, and isolate 24/H/91/BCY as wild type (Table 4). The RRFLP technique correctly differentiated each vaccine and agreed with sequencing results presented as sequence similarities in Table 4 and illustrated in FIG. 3.

Polymorphic sites stability and reproducibility of RRFLP. The stability of the informative polymorphic sites and the reproducibility of the RRFLP assay were assessed using trachea samples collected from CEO and TCO vaccinated chickens, and chickens exposed to vaccinated birds at different days post vaccination. Results are summarized in Table 5. RRFLP analysis of trachea samples from CEO vaccinated and CEO contact-exposed chickens produced ΔC_{T}≧1 for the Alw I digestion reactions, while tracheal samples from TCO vaccinated and TCO contact exposed chickens produced ΔC_{T}≧1 for Ava I digestion reactions (Table 5).

### Discussion

Although minor antigenic differences exist among laryngotracheitis viral strains, these minor antigenic differences have not been sufficient to separate strains into serotypes (Guy and Garcia, “Infectious laryngotracheitis virus,” in: Saif, Y. M., Glisson, J. R., Fadly, A. M., McDougald, L. R., Nolan, L. K., Swayne, D. E. (Eds.), Diseases of Poultry 12^{th} edition, 2007. Blackwell Publishing Inc., Ames, Iowa). Therefore strain differentiation has been mainly accomplished by the distinction of viral genotypes using PCR-RFLP and sequencing analysis of multiple viral genes (Kirkpartick et al., 2006, Avian Dis; 50:28-34; Ojkic et al., 2006, Avian Pathol; 35 (4):286-292; Oldoni et al., 2007, Avian Pathol; 36:167-176; and Creelan et al., 2006, Avian Pathol; 5 (2):173-179).

The identification of single nucleotide polymorphism (SNP) has allowed the differentiation of closely related strains in the United Kingdom (Creelan et al., 2006, Avian Pathol; 5 (2):173-179) and Korea (Han and Kim, 2001, Vet. Microbiol; 4:321-331). In the United States ILT strains have shown to be closely related to the CEO vaccine (Oldoni et al., 2007, Avian Pathol; 36:167-176) and SNPs have been identified that allowed differentiation of these strains (Oldoni et al., 2007, Avian Pathol; 36:167-176). In this study a rapid analysis of pre-determined SNPs by RRFLP permitted a clear differentiation of ILT live attenuated vaccines (CEO and TCO). Genotyping results obtained by RRFLP for ten ILT isolates, six CEO vaccines, and the TCO vaccine were confirmed by sequencing analysis. In addition, CEO and TCO vaccines were detected and identified directly form tracheal swabs from vaccinated birds, and from birds in contact exposure to vaccinate. The RRFLP assay can be used to detect and differentiate the presence of any DNA molecule that contains an informative restriction enzyme site located between two viable PCR primer sites. A pre-requisite for developing an RRFLP assay is the creation of a robust sequence database that allows for the discovery of informative SNPs with restriction enzyme sites with a desired identification, differentiation, or genotyping scheme for specific DNA molecules.

It should be noted that any existing RFLP based detection/differentiation method that utilizes an informative (unique and specific) restriction enzyme site between two viable PCR primer sites could be easily converted to the RRFLP technique avoiding the use of more ambiguous and tedious gel based method for the differentiation of DNA molecules.

The RRFLP assay of the present invention is well suited for use as a novel diagnostic method for the detection/differentiation of pathogens, as exemplified in this communication for the differentiation of ILTV isolates, and it can be utilized in numerous ways with all organisms that use DNA as their genetic instructions. RRFLP uses inexpensive, long shelf-life restriction enzymes, instead of expensive, short shelf-life probes. Further, the RRFLP assay may be used in a combination of compatible restriction enzymes in fast digestion reactions (10 minute digest) together with the real-time PCR reaction in a one step procedure this will greatly accelerate, simplify and reduce the cost of the assay.

Sequence analysis of ILTV vaccines revealed an informative polymorphic site in the 5′ non-coding region of the infected cell protein (ICP4) present in the tissue culture origin (TCO) and chicken embryo origin (CEO) attenuated vaccines recognized by restriction enzymes AvaI and AlwI, respectively. These two informative polymorphic sites were used in a RRFLP assay to rapidly genotype ILTV attenuated live vaccines. In the RRFLP procedure, DNA is digested with restriction enzymes targeting the informative polymorphic site and then used as template in a real-time polymerase chain reaction (PCR) with primers flanking this region. The analysis of the ΔC_{T} values obtained from digested and undigested template DNA provides the genotype of the DNA molecule.

TABLE 2
Infectious laryngotracheitis virus outbreak related isolates.

| Isolates^{a} | Age^{b} | Vaccination |
|---|---|---|
| 24/H/91/BCK | 183 to 548 | NV^{c} |
| 9/C/97/BR | 56 | NV |
| 10/C/97/BR | 53 | CEO |
| 23/H/01/BBR | 392 | NV |
| 13/E/03/BBR | 441 | TCO |
| 15/E/03/BR | 35 | NV |
| 26/I/03/BR | 40 | NV |
| 11/C/05/BR | 42 | NV |
| 21/G/05/BR | 42 | NV |
| 314/K/BR/04 | 33 | NV |

^{a}Sample number/State/Year of isolation/Bird type: States of origin are indicated by letters (C, E, G, H, I) - each isolate with the same letter originated in the same state; bird type: from commercial poultry broiler (BR), broiler breeder (BBR), from backyard flock (BCY)
^{b}Bird age expressed in days;
^{c}Flocks non-vaccinated against ILTV.

TABLE 3
ILTV ICP4 gene primer sequences.

| | | Nucleotide |
|---|---|---|
| Primer name | Sequence 5′ to 3′ | positions^{a} |
| ICP4 | ACG GTA ATG GTA TGC TGG G | 1807-1825 |
| non-coding^{b} | (SEQ ID NO: 5) | |
| ICP4 | CTC ACA GCG GTT GTT TTC TC | 3052-3033 |
| coding^{b} | (SEQ ID NO: 6) | |
| ICP4 poly- | TAC TAC TCC CCA CCA GAA AG | 2389-2408 |
| morphic | (SEQ ID NO: 7) | |
| site F^{c} | | |
| ICP4 poly- | CGT CGA GGA ATC AGA GGA CAT | 2534-2514 |
| morphic | (SEQ ID NO: 8) | |
| site R^{c} | | |

^{a}Primer position according to ICP4 sequence accession number L32139
^{b}Sequencing primers
^{c}Real-time PCR primers

TABLE 4
Reverse RFLP and Sequencing analysis ILTV vaccines (CEO and TCO) and outbreak related isolates

| | ReTi— | RRFLP | RRFLP | RRFLP | | | RRFLP | Sequencing |
|---|---|---|---|---|---|---|---|---|
| Vaccine/Isolate | PCR C_{T}^{c} | Undigested C_{T} | Alw I C_{T} | Ava I C_{T} | ΔC_{T} Alw I | ΔC_{T} Ava I | Interpretation^{d} | Analysis^{f} |
| CEO VAC 1^{a} | 25.32 | 30.83 | 33.87 | 29.97 | 3.04 | −0.86 | CEO | CEO (100%) |
| CEO VAC 2^{a} | 24.94 | 29.99 | 34.70 | 29.93 | 4.71 | −0.06 | CEO | CEO (100%) |
| CEO VAC 3^{a} | 25.08 | 29.98 | 34.92 | 29.85 | 4.94 | −0.13 | CEO | CEO (100%) |
| CEO VAC 4^{a} | 24.51 | 29.98 | 34.95 | 29.92 | 4.97 | −0.06 | CEO | CEO (100%) |
| CEO VAC 5^{a} | 23.21 | 30.86 | 34.95 | 30.37 | 4.09 | −0.49 | CEO | CEO (100%) |
| CEO VAC 6^{a} | 23.38 | 30.39 | 34.79 | 29.92 | 4.40 | −0.47 | CEO | CEO (100%) |
| CEO VAC 4 (p3)^{b} | 15.51 | 18.32 | 22.80 | 18.71 | 4.48 | 0.39 | CEO | CEO (100%) |
| TCO VAC | 19.07 | 24.48 | 25.27 | 29.16 | 0.79 | 4.68 | TCO | TCO (100%) |
| TCO VAC (p3)^{b} | 18.70 | 24.00 | 24.70 | 27.34 | 0.70 | 3.34 | TCO | TCO (100%) |
| 24/H/91/BCY | 22.44 | 25.77 | 25.94 | 25.56 | 0.17 | −0.21 | WT^{e} | TCO (99.8%) |
| 9/C/97/BR | 12.47 | 17.14 | 21.86 | 17.26 | 4.72 | 0.12 | CEO | CEO (100%) |
| 10/C/97/BR | 12.68 | 15.68 | 18.94 | 15.72 | 3.26 | 0.04 | CEO | CEO (100%) |
| 23/H/01/BBR | 23.70 | 27.43 | 28.86 | 26.77 | 1.43 | −0.66 | CEO | CEO (100%) |
| 13/E/03/BBR | 23.54 | 25.97 | 26.12 | 30.72 | 0.15 | 4.75 | TCO | TCO (100%) |
| 15/E//03/BR | 20.14 | 22.90 | 27.34 | 23.20 | 4.44 | 0.30 | CEO | CEO (100%) |
| 26/I/03/BR | 18.27 | 20.76 | 26.15 | 21.14 | 5.39 | 0.38 | CEO | CEO (100%) |
| 11/C/05/BR | 11.84 | 18.14 | 22.46 | 18.37 | 4.32 | 0.23 | CEO | CEO (100%) |
| 21/G/05/BR | 21.09 | 27.50 | 30.50 | 27.25 | 3.00 | −0.25 | CEO | CEO (100%) |
| 314/K/BR/04 | 14.23 | 19.39 | 23.98 | 19.40 | 4.59 | 0.01 | CEO | CEO (100%) |

^{a}Dilutions of the vaccines (1:100) were utilized for the RRFLP analysis;
^{b}Third passages in chicken kidney cells of CEO and TCO vaccines;
^{c}Callison et al., 2007, J. Virol. Meth; 139: 31-38;
^{d}A sample with ΔCT values ≦1 for either enzyme was genotyped as wild-type (WT); A sample with a ΔCT of ≧1 for Alw I and a ΔCt ≦1 for Ava I was genotyped as chicken origin embryo (CEO); a sample with a ΔCT ≦1 for Alw I and a ΔCT ≧1 for Ava I was genotyped as tissue culture origin (TCO);
^{e}Wild type strain;
^{f}Sequence analysis of nucleotides 2039 to 2950 of the ICP4 gene (Accession number L32139), indicated the strain with the highest nucleotide sequence similarity and in parenthesis the percentage of sequence similarity.

TABLE 5
Reverse RFLP analysis on tracheal samples collected from CEO and TCO vaccinated and contact-expose birds

| | ReTi— | RRFLP | RRFLP | RRFLP | | | |
|---|---|---|---|---|---|---|---|
| | PCR C_{T}^{b} | Undigested C_{T} | Alw I C_{T} | Ava I C_{T} | ΔC_{T} Alw I | ΔC_{T} Ava I | Interpretation^{e} |
| CEO Vaccinated | | | | | | | |
| 2^{a} | 27.48 | 32.39 | 37.14 | 32.87 | 4.75 | 0.48 | CEO |
| 4 | 22.32 | 28.29 | 34.01 | 29.15 | 5.72 | 0.86 | CEO |
| 5 | 26.41 | 33.12 | 38.87 | 32.87 | 5.75 | −0.25 | CEO |
| CEO Contact exposed | | | | | | | |
| 5^{a} | 30.14 | 40.22 | 43.10 | 40.76 | 2.88 | 0.54 | CEO |
| 6 | 0^{c} | — | — | — | — | — | — |
| 7 | 0 | — | — | — | — | — | — |
| 8 | 28.47 | 38.72 | 41.93 | 37.94 | 3.21 | −0.78 | CEO |
| 9 | 28.18 | 38.67 | 43.21 | 38.10 | 4.54 | −0.57 | CEO |
| 10 | 0 | — | — | — | — | — | — |
| TCO Vaccinated | | | | | | | |
| 4 | 32.27 | —^{d} | — | — | — | — | — |
| 6 | 26.01 | 33.40 | 34.10 | 38.20 | 0.58 | 5.56 | TCO |
| 7 | 36.12 | — | — | — | — | — | — |
| TCO Contact expose | | | | | | | |
| 9 | 26.33 | 36.24 | 36.50 | 40.37 | 0.26 | 4.13 | TCO |
| 18 | 29.79 | 39.03 | 38.75 | 43.50 | −0.28 | 4.47 | TCO |

^{a}Days post-vaccination;
^{b}Callison et al., 2007;
^{c}sample with no viral DNA detected;
^{d}Samples with not sufficient viral DNA (Ct > 31.00 Re—Ti PCR) for RRFLP analysis;
^{e}RRFLP interpretation - a sample with ΔCt values ≦1 for either enzyme was genotyped as a wild-type (WT) strain, a sample with a ΔCt value for Alw I ≧1 and a ΔCt value for Ava I ≦1 was genotyped as a CEO like isolate, a sample with a ΔCt value for Alw I ≦1 and a ΔCt value for Ava I ≧1 was genotyped as TCO like strain.

### Example 3

### Testing Stability of the ICP4 Polymorphic Site and Reproducibility of the Reverse Restriction Fragment Length Polymorphism Assay

In this example, the quality of the novel RRFLP assay was assessed by analyzing the stability of the polymorphic ICP4 site and by examining the reproducibility of the assay itself. In order to evaluate the reproducibility of the RRFLP analysis, two separate experiments with CEO and TCO vaccinated, and vaccine contact exposed chickens were performed.

### Materials and Methods

Animals. Ninety-six white leghorn specific pathogen free (SPF) chickens were obtained from Merial (Gainesville, Ga.) for each experiment. Chickens were housed in stainless steel cages in the isolation room with filtered-air and positive-pressure at the Poultry Diagnostic and Research Center (PDRC, Athens, Ga.), and fed a standard diet and water ad libitum.

Vaccination Experiments. At four weeks of age, birds were divided in four groups of twenty-four chickens per cage, 12 of which were vaccinated, and 12 were contact-exposed to the vaccinated birds. Wing bands were used in to identify contact-exposed birds. Chickens were vaccinated by eye-drop with the TCO and CEO live attenuated vaccine during two separate experiments using the recommended dose (33 μl per chicken). Larynx/trachea and eye conjunctiva swabs were collected from two vaccinated and two contact-exposed chickens at different time pointes post-vaccination. Eye conjunctiva swabs were collected from both eyes and resuspended in 1 ml of sterile phosphate buffered saline solution (PBSS) containing antibiotic-antimycotic (Gibco, Grand Island, N.Y.) and 2% newborn calf serum (Gibco Grand Island, N.Y.). Chickens were euthanized by CO₂ gas inhalation (Institutional Animal Care and Use Committee). During necropsy, the larynx and the trachea were dissected. The larynx and trachea were open longitudinally and the inside epithelium was scraped. Larynx and trachea scrapings were resuspended in 1 ml of PBSS. Both eye conjunctiva and tracheal samples were stored at −80° C. until further PCR processing.

### Results

The stability of the polymorphic site within the ICP4 gene (FIG. 4) was assessed by testing different commercially available ILTV vaccines from the bottle and vaccines after several in vivo and in vitro passages by RRFLP and sequence analysis (Table 6). In total, six different CEO vaccines, four different CEO passages, one TCO vaccine, and five different passages were analyzed. The polymorphic site was stable in each vaccine tested.

The stability of the informative region upstream of the ICP4 gene was further assessed using samples collected from CEO and TCO vaccinated birds. Eye conjunctiva and trachea samples were collected from CEO and TCO vaccinated and contact exposed chickens at different days post vaccination. The results for eye conjunctiva samples are summarized in Table 7.

TABLE 6
Reproducibility of Reverse RFLP on commercially available
ILTV vaccines and vaccine in vivo and in vitro passages

| | | | | Sequencing |
|---|---|---|---|---|
| | ΔCt value | ΔCt value | RRFLP inter- | interpreta- |
| Isolate | for AlwI^{a} | for AvaI^{b} | pretation^{c} | tion^{d} |
| CEO 1 | 5.42 | 0.36 | CEO | CEO |
| CEO 2 | 4.01 | 0.04 | CEO | CEO |
| CEO 3 | 3.00 | 0.49 | CEO | CEO |
| CEO 4 | 4.13 | 0.09 | CEO | CEO |
| CEO 5 | 4.20 | −0.22 | CEO | CEO |
| CEO 6 | 3.40 | −0.32 | CEO | CEO |
| CEO 4 (5 DPV)^{e} | 6.13 | −0.41 | CEO | CEO |
| CEO 4 (9 DPV)^{e} | 6.25 | −0.09 | CEO | CEO |
| CEO 4 (p1)^{f} | 3.36 | −0.80 | CEO | CEO |
| CEO 4 (p6)^{f} | 4.48 | −0.53 | CEO | CEO |
| TCO | 0.16 | 3.63 | TCO/USDA | TCO/USDA |
| TCO 5 (DPV)^{e} | 0.43 | 3.74 | TCO/USDA | ND^{g} |
| TCO 9 (DPV)^{e} | 0.18 | 4.33 | TCO/USDA | ND |
| TCO (p1)^{f} | 0.26 | 3.59 | TCO/USDA | ND |
| TCO (p2)^{f} | 0.19 | 3.54 | TCO/USDA | ND |
| TCO (p3)^{f} | 0.41 | 3.80 | TCO/USDA | ND |

^{a}ΔCt value for Alw I = (Ct value of sample DNA cut with Alw I) − (Ct value of uncut DNA sample)
^{b}ΔCt value for Ava I = (Ct value of sample DNA cut with Ava I) − (Ct value of uncut DNA sample)
^{c}RRFLP interpretation - a sample with ΔCt values ≦1 for either enzyme was interpreted as wild-type (WT); a sample with a ΔCt value for Alw I ≧1 and a ΔCt value for Ava I ≦1 was interpreted as chicken origin embryo (CEO) like isolate; a sample with a ΔCt value for Alw I ≦1 and a ΔCt value for Ava I ≧1 was interpreted as USDA or tracheal culture origin (TCO) like strain (USDA/TCO)
^{d}Sequence interpretation - as determined by the phylogenetic relationship of the ICP4 gene fragment analyzed.
^{e}Vaccine recovered from vaccinated birds 5 and 9 days post vaccination (DPV).
^{f}Vaccine after consecutive passages (p #) in chicken embryo kidney (CEK) cells.
^{g}Not done.

RRFLP analysis accurately and reproducibly identified each vaccine type. The RRFLP analysis was reproducible and consistent for each type of vaccine at the different time point tested. RRFLP analysis of CEO vaccinated and contact-exposed chickens produced ΔC_{T}≧1 for Alw I digestion reactions and for TCO vaccinated and contact exposed chickens ΔC_{T}≧1 were observed for Ava I digestions (Table 7). Similar RRFLP results were observed for tracheal swab samples collected from vaccinated and contact exposed chickens.

The limit of detection for the RRFLP assay was determined by testing 10-fold serial dilutions of DNA extracted from a CEO vaccine. The dilution representing 10³ genomic copies (as determined by Real-Time PCR ILTV assay) was the last dilution positive for the RRFLP assay.

TABLE 7
Reverse RFLP analysis on eye conjunctiva samples collected
from CEO and TCO vaccinated and contact-exposed birds

| | ReTi— | RRFLP | RRFLP | RRFLP | ΔC_{T} | ΔC_{T} | |
|---|---|---|---|---|---|---|---|
| | PCR C_{T}^{b} | Undigested C_{T} | Alw I C_{T} | Ava I C_{T} | Alw I | Ava I | Interpretation^{c} |
| CEO vaccinated^{a} | | | | | | | |
| 4 | 19.77 | 24.81 | 29.24 | 24.14 | 4.43 | −0.67 | CEO |
| 6 | 24.19 | 33.11 | 37.93 | 32.78 | 4.82 | −0.33 | CEO |
| CEO contact-exposed^{a} | | | | | | | |
| 9 | 24.44 | 30.22 | 36.01 | 30.84 | 5.79 | 0.62 | CEO |
| TCO vaccinated^{a} | | | | | | | |
| 4 | 25.44 | 33.40 | 34.10 | 38.20 | 0.70 | 4.80 | TCO |
| 6 | 25.70 | 32.84 | 31.87 | 36.16 | −0.97 | 3.32 | TCO |
| TCO contact-exposed^{a} | | | | | | | |
| 9 | 23.57 | 30.79 | 31.12 | 35.78 | 0.33 | 4.99 | TCO |

^{a}Days post-vaccination;
^{b}Callison et al., 2007;
^{c}RRFLP interpretation - a sample with ΔC_{T} values ≦1 for either enzyme was genotyped as a wild-type (WT) strain, a sample with a ΔC_{T} value far Alw I ≧1 and a ΔC_{T} value for Ava I ≦1 was genotyped as a CEO like isolates, a sample with a ΔC_{T} value for Alw I ≦1 and a ΔC_{T} value for Ava I ≧1 was genotyped as TCO like isolate.

### Example 4

### Genotyping of Infectious Laryngotracheitis Virus in Clinical Samples

In this example, tracheas were collected from flocks affected by three recent ILTV outbreaks in broiler flocks and genotyped.

### Materials and Methods

Clinical Samples. A total of 32 cases from three independent outbreaks were processed. Briefly, an average of three tracheas were received per case and processed separately. The mucosa of the trachea and larynx were scraped with a scalpel and the scrapings were resuspended in 1 ml of sterile 1×PBS containing 100 μg/ml of gentamicin (Invitrogen, Carlsbad, Calif.) and 5 μg/ml of amphotericin B (Invitrogen, Carlsbad, Calif.). Samples were vortexed and stored at −80° C. until further analysis.

Genotyping of clinical samples by RRFLP assay. Tracheal samples from three recent outbreaks of ILTV were analyzed by RRFLP and the results confirmed by sequence analysis of the ICP4 gene. All samples were genotyped as CEO-like virus by both methods (Table 8).

TABLE 8
Reverse RFLP and sequencing analysis on
tracheal samples from ILTV outbreaks

| | | | | Sequencing |
|---|---|---|---|---|
| Tracheal | ΔCt value | ΔCt value | RRFLP inter- | interpreta- |
| Samples | for Alw I^{a} | for Ava I^{b} | pretation^{c} | tion^{d} |
| Outbreak 1A | 5.37 | 0.32 | CEO | CEO |
| 1B | 4.28 | 0.01 | CEO | CEO |
| 1C | 9.88 | 0.45 | CEO | CEO |
| 1D | 7.32 | 0.42 | CEO | CEO |
| 1E | 7.28 | 0.48 | CEO | CEO |
| 1F | 8.05 | 0.12 | CEO | CEO |
| 1G | 5.61 | 0.11 | CEO | CEO |
| 1H | 5.55 | 0.10 | CEO | CEO |
| 1I | 7.75 | 0.46 | CEO | CEO |
| 1J | 5.60 | 0.45 | CEO | CEO |
| 1K | 6.60 | 0.76 | CEO | CEO |
| 1L | 7.19 | 0.61 | CEO | CEO |
| 1M | 5.37 | 0.32 | CEO | CEO |
| 1N | 6.11 | 0.41 | CEO | CEO |
| 1O | 5.00 | 0.20 | CEO | CEO |
| 1P | 6.18 | −0.16 | CEO | CEO |
| 1Q | 4.89 | −0.38 | CEO | CEO |
| 1R | 4.37 | −0.04 | CEO | CEO |
| 1S | 6.76 | 0.15 | CEO | CEO |
| 1T | 4.21 | 0.69 | CEO | CEO |
| 1U | 6.70 | 0.39 | CEO | CEO |
| 1V | 4.85 | 0.23 | CEO | CEO |
| Outbreak 2A | 5.53 | 0.14 | CEO | CEO |
| 2B | 6.88 | −0.05 | CEO | CEO |
| 2C | 7.04 | 0.48 | CEO | CEO |
| 2D | 5.24 | −0.36 | CEO | CEO |
| Outbreak 3A | 5.91 | 0.03 | CEO | CEO |
| 3B | 4.60 | −0.40 | CEO | CEO |
| 3C | 2.67 | −0.33 | CEO | CEO |
| 3D | 5.34 | 0.27 | CEO | CEO |
| 3E | 4.98 | 0.25 | CEO | CEO |
| 3F | 5.60 | 0.40 | CEO | CEO |

^{a}ΔCt value for Alw I = (Ct value of sample DNA cut with Alw I) − (Ct value of uncut DNA sample).
^{b}ΔCt value for Ava I = (Ct value of sample DNA cut with Ava I) − (Ct value of uncut DNA sample)
^{c}RRFLP interpretation - a sample with ΔCt values ≦1 for either enzyme was interpreted as wild-type (WT); a sample with a ΔCt value for Alw I ≧1 and a ΔCt value for Ava I ≦1 was interpreted as chicken origin embryo (CEO) like isolate; a sample with a ΔCt value for Alw 1 ≦1 and a ΔCt value for Ava I ≧1 was interpreted as USDA or tracheal culture origin (TCO) like strain (USDA/TCO)
^{d}Sequence interpretation - as determined by the phylogenetic relationship of the ICP4 gene fragment analyzed.

The complete disclosure of all patents, patent applications, and publications, and electronically available material (including, for instance, nucleotide sequence submissions in, e.g., GENBANK (GENBANK is a registered trademark of the U.S. Department of Health and Human Services, National Institutes of Health) and RefSeq, and amino acid sequence submissions in, e.g., SwissProt, PIR, PRF, PDB, and translations from annotated coding regions in GENBANK and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified. Unless otherwise specified, “a,” “an,” “the,” and “at least one” are used interchangeably and mean one or more than one.

For any method disclosed herein that includes discrete steps, the steps may be conducted in any feasible order. And, as appropriate, any combination of two or more steps may be conducted simultaneously.

SEQUENCE LISTING FREE TEXT

| | |
|---|---|
| SEQ ID NO: 1 | Basepairs 2392 to 2534 of the ICP4 gene sequence as |
| | found in the chicken embryo origin (CEO) vaccine. |
| SEQ ID NO: 2 | Basepairs 2392 to 2534 of the ICP4 gene sequence as |
| | found in the tissue culture origin (TCO) vaccine. |
| SEQ ID NO: 3 | Basepairs 2392 to 2534 of the ICP4 gene sequence as |
| | found in broiler (9/C/97/BR) and broiler/breeder |
| | (23/H/01/BBR) isolates, identified as BR/BBR. |
| SEQ ID NO: 4 | Basepairs 2392 to 2534 of the ICP4 gene sequence as |
| | found in the backyard flock isolate(24/H/91/BCK) |
| | identified as BCK. |
| SEQ ID NO: 5 | ILTV ICP4 non-coding region synthetic |
| | oligonucleotide gene primer. |
| SEQ ID NO: 6 | ILTV ICP4 coding region synthetic oligonucleotide |
| | gene primer. |
| SEQ ID NO: 7 | ILTV ICP4 polymorphic site forward synthetic |
| | oligonucleotide gene primer. |
| SEQ ID NO: 8 | ILTV ICP4 polymorphic site reverse synthetic |
| | oligonucleotide gene primer. |

What is claimed is:

1. A method of detecting the presence of a recognition site for a restriction enzyme in a nucleotide sequence, the method comprising:
digesting all or a portion of a sample comprising the nucleotide sequence with the restriction enzyme;
performing real-time polymerase chain reaction (PCR) on the sample digested with the restriction enzyme with an oligonucleotide primer pair that flanks the restriction enzyme recognition site;
determining the Ct value of the sample digested with the restriction enzyme;
comparing the Ct value of the sample digested with the restriction enzyme to the Ct value from a control sample not digested with the restriction enzyme;
calculating a ΔCt value, wherein a ΔCt value is the Ct value of the sample digested with the restriction enzyme minus the Ct value of a control sample not digested with the restriction enzyme;
wherein a ΔCt value ≧ about +1 indicates that the nucleotide sequence is digested by the restriction enzyme at a recognition site located between the oligonucleotide primer pair.

2. The method of claim 1 wherein separate portions of the sample are digested with different restriction enzymes and a separate ΔCt value is calculated for each separate portion.

3. The method of claim 1 used in the detection and/or differentiation of infectious laryngotracheitis virus (ILTV) strains.

4. The method of claim 3, wherein a portion of the sample is digested with the restriction enzyme Alw 1.

5. The method of claim 3, wherein a portion of the sample is digested with the restriction enzyme Ava 1.

6. The method of claim 3, wherein a portion of the sample is digested with the restriction enzyme Ava I and a portion of the sample is digested with the restriction enzyme Alw 1.

7. The method of claim 6, wherein the presence of a restriction enzyme site for Ava I and the absence of a restriction enzyme site for Alw 1 indicates the ILTV is the tissue culture origin (TCO) vaccine virus, and wherein the presence of a restriction enzyme site for Alw I and the absence of a restriction enzyme site for Ava 1 indicates the ILTV is the chicken embryo origin (CEO) vaccine virus.

8. The method of claim 3, wherein the oligonucleotide primer pair flanks about nucleotide 60 to about nucleotide 80 of the ILTV ICP4 gene promoter sequence.

9. The method of claim 3, wherein the oligonucleotide primer pair flanks nucleotide positions 2039 to 2950 of the ILTV ICP4 gene (Accession No. L32139).

10. The method of claim 3, wherein the oligonucleotide primer pair flanks nucleotide positions 2392 to 2534 of the ILTV ICP4 gene (Accession No. L32139).

11. The method of claim 3, wherein the oligonucleotide primer pair is SEQ ID NO:7 and SEQ ID NO:8.

12. A method of detecting infectious laryngotracheitis virus (ILTV), the method comprising digesting a nucleotide sample with the restriction enzymes Alw 1 and/or Ava 1 and detecting the presence or absence of recognition sites for the restriction enzymes Alw 1 and/or Ava 1 within about nucleotide 60 to about nucleotide 80 of the ILTV ICP4 gene promoter sequence.

13. An oligonucleotide primer pair that flanks about nucleotide 60 to about nucleotide 80 of the ICP4 gene promoter sequence of the infectious laryngotracheitis virus (ILTV) ICP4 gene (Accession No. L32139).

14. The oligonucleotide primer pair of claim 13, wherein the forward primer comprises SEQ ID NO:7.

15. The oligonucleotide primer pair of claim 13, wherein the reverse primer comprises SEQ ID NO:8
